(19) United States
(12) Patent Application Publication
(10) Pub. No.: US 2025/0129153 A1
(43) Pub. Date: Apr. 24, 2025

(54) CELL-SURFACE ANTIBODY TO A SPECIFIC BIOMARKER OF PANCREATIC BETA-CELLS

(71) Applicants: THE JOHNS HOPKINS UNIVERSITY, Baltimore, MD (US); THE REGENTS OF THE UNIVERSITY OF COLORADO, A BODY CORPORATE, Denver, CO (US)

(72) Inventors: Dax Fu, Short Hills, NJ (US); Liping Yu, Centennial, CO (US); Zheng Guo, Baltimore, MD (US)

(21) Appl. No.: 18/685,045

(22) PCT Filed: Aug. 18, 2022
(86) PCT No.: PCT/US2022/075156

§ 371 Date: Feb. 20, 2024

Related U.S. Application Data
(60) Provisional application No. 63/388,005, filed on Jul. 11, 2022.
(60) Provisional application No. 63/235,237, filed on Aug. 20, 2021.

(51) Int. Cl. C07K 16/28; A61K 47/68; A61K 51/10

(52) U.S. Cl. CPC C07K 16/28; A61K 47/6849; A61K 51/1027; A61K 51/1093; A61K 2123/00; C07K 2317/565; C07K 2317/77

(57) ABSTRACT

The present invention relates to the field of immunology. More specifically, the present invention provides methods and compositions directed to the use of antibodies to the pancreatic zinc transporter, ZnT8. In particular embodiments, the anti-ZnT8 antibodies specifically bind the transmembrane domain of ZnT8. In more specific embodiments, the anti-ZnT8 antibodies specifically the extracellular surface of the transmembrane domain.

### CROSS-REFERENCE TO RELATED APPLICATIONS

This application claims priority to U.S. Provisional Patent Application No. 63/235,237, filed on Aug. 20, 2021, and U.S. Provisional Patent Application No. 63/388,005, filed on Jul. 11, 2022, which are incorporated herein by reference in their entireties.

### FEDERALLY SPONSORED RESEARCH OR DEVELOPMENT

This invention was made with government support under grant DK125746 and DK123435 awarded by the National Institutes of Health. The government has certain rights in the invention.

### SEQUENCE LISTING

This application contains a Sequence Listing that has been submitted electronically as an XML file named 448070440WO1.xml. The XML file, created on Aug. 17, 2022, is 54,472 bytes in size. The material in the XML file is hereby incorporated by reference in its entirety.

### TECHNICAL FIELD

Provided herein are materials and methods relating to the fields of immunology and diabetes. More specifically, provided herein are methods and compositions directed to the use of antibodies to the pancreatic zinc transporter, ZnT8.

### BACKGROUND

Pancreatic β-cells as professional secretory cells provide the sole source of insulin in the human body to control blood glucose levels. While β-cells have evolved large dynamic capacities of insulin production in response to glucose fluctuations, they are poorly equipped to cope with islet inflammation and metabolic stress underlying β-cell autoimmune vulnerability in type-1 diabetes (1), and β-cell failure and loss in type-2 diabetes (2). Hence, primary β-cell defects lie at the heart of susceptibility to both forms of diabetes. A better understanding of diabetes pathogenesis and evaluation of therapeutic interventions require exact monitoring of the fate of β-cells under disease and therapy conditions. However, routine tests such as measurements of the insulin/C-peptide level, fasting blood glucose level and oral glucose tolerance do not provide adequate information about the mass and function of insulin-producing β-cells in the pre-clinical phase of diabetes and after receiving intervention therapy. Cell surface biomarkers directly linked to the insulin secretory biology with a high cell-surface density are valuable targets for the development of cell-surface monoclonal antibodies (mAbs) applicable to noninvasive monitoring of 3-cell functions and drug delivery.

### SUMMARY

The dysfunction and loss of insulin-producing β-cells in pancreatic islets are primary causes of diabetes mellitus, but neither in vivo monitoring of β-cell mass and function nor methods for targeted drug delivery have yet been developed. Insulin production and storage in β-cells are functionally coupled with cellular zinc enrichment, which is controlled by the hyperexpression of an islet-specific zinc transporter-8 (ZnT8). Described herein are autoreactive antibodies (mAb43) with a subnanomolar binding affinity and conformation specificity for an extracellular epitope of ZnT8. Glucose stimulation increased ZnT8-mAb43 binding on the extracellular cell surface, enabled the use of mAb43 to isolate j-cells from single-cell suspensions of whole pancreas and to guide islet-homing of a fluorescent tag in mice following systemic administration. In some embodiments, the autoreactive antibodies can target j-cell surface ZnT8 for in vivo delivery of imaging payloads and antibody-drug conjugates.

Provided herein are antibodies or antigen-binding fragments thereof that specifically binds to three extracellular loops of the transmembrane domain of Zinc Transporter-8 (ZnT8). In some embodiments, the three extracellular loops of ZnT8 comprise amino acids 95-99, 169-175 and 242-245 of SEQ ID NO: 31.

In some embodiments, the antibody or antigen-binding fragment thereof comprises: (a) heavy chain complementarity determining regions (CDRs) 1, 2 and 3 comprising SEQ ID NOs: 3-5, respectively; and (b) light chain CDRs 1, 2 and 3 comprising SEQ ID NOs: 8-10, respectively. In some embodiments, the antibody or antigen-binding fragment thereof comprises at least one conservative amino acid substitution within one or more of SEQ ID NOs: 3-5 and/or at least one conservative amino acid substitution within one or more of SEQ ID NOs: 8-10. In some embodiments, the antibody or antigen-binding fragment thereof comprises: (a) a heavy chain variable region sequence having at least 90% sequence identity to SEQ ID NO: 2 or SEQ ID NO: 19; and (b) a light chain variable region sequence having at least 90% sequence identity to SEQ ID NO: 7. In some embodiments, the heavy chain variable region sequence has at least 95% sequence identity to SEQ ID NO: 2 or SEQ ID NO: 19, and the light chain variable region sequence has at least 95% sequence identity to SEQ ID NO: 7. In some embodiments, the antibody or antigen-binding fragment thereof comprises: (a) a heavy chain variable region sequence comprising SEQ ID NO: 2 or SEQ ID NO: 19; and (b) a light chain variable region sequence comprising SEQ ID NO: 7.

In some embodiments, the fragment comprises a Fab, Fab′, F(ab′)2, Fab¹-SH, Fv, diabody, linear antibody or single-chain variable fragment (scFv). In some embodiments, the heavy chain constant region is of the immunoglobulin G1 (IgG1) isotype. In some embodiments, the antibody or antigen-binding fragment thereof is a humanized or chimeric antibody. In some embodiments, the antibody or antigen-binding fragment thereof is conjugated to a therapeutic agent. In some embodiments, the antibody or antigen-binding fragment thereof is conjugated to an imaging agent.

Also provided herein are pharmaceutical compositions comprising a therapeutically effective amount of any of the antibody or antigen-binding fragment thereof described herein.

Also provided herein are nucleic acid molecules encoding any of the antibodies or antigen-binding fragments described herein. Also provided herein are vectors comprising any of the nucleic acids described herein. Also provided herein are host cells comprising any of the vectors described herein.

Also provided herein are methods for producing an antibody drug-conjugate that specifically binds three extracellular loops of the transmembrane domain of ZnT8, the method comprising: (a) culturing any of the host cells described herein under conditions suitable for production of the antibody; and (b) conjugating the antibody to a therapeutic agent.

Also provided herein are methods for producing an antibody imaging agent-conjugate that specifically binds three extracellular loops of the transmembrane domain of ZnT8, the method comprising: (a) culturing any of the host cells described herein under conditions suitable for production of the antibody; and (b) conjugating the antibody to an imaging agent.

Also provided herein are methods for treating a disease or condition associated with ZnT8 in a subject, the method comprising administering to the subject any of the antibodies or antigen-binding fragments described herein. In some embodiments, the disease or condition comprises type 1 or type 2 diabetes.

Also provided herein are methods for detecting pancreatic beta cells in vivo, the method comprising administering any of the antibodies or antigen-binding fragments described herein to a subject and detecting the imaging agent conjugated to the antibody or antigen-binding fragment thereof. In some embodiments, the detecting step comprises positron emission tomography (PET), single-photon emission computed tomography (SPECT)/CT imaging, nuclear magnetic resonance (NMR) spectroscopy or near-infrared (NIR) optical imaging.

In some embodiments, the antibody or antigen-binding fragment comprises a single chain variable fragment (scFv) comprising (a) a heavy chain variable region sequence comprising SEQ ID NO: 2 or SEQ ID NO: 19; and (b) a light chain variable region sequence comprising SEQ ID NO: 7. In some embodiments, the heavy chain variable region sequence comprises at least one conservative amino acid substitution within SEQ ID NO: 2 or SEQ ID NO: 19; and (b) the light chain variable region sequence comprises at least one conservative amino acid substitution within SEQ ID NO: 7.

In some embodiments, the imaging agent is a radiometal. In some embodiments, the imaging agent is a radiometal and the detecting step comprises PET. In some embodiments, the radiometal is selected from the group consisting of ⁶⁴Cu, ⁶⁷Cu, ⁶⁸Ga, ⁶⁰Ga, ⁸⁹Zr, ⁸⁶Y, and ^{94m}Tc. In some embodiments, the imaging agent is a radiometal and the detecting step comprises SPECT. In some embodiments, the radiometal is selected from the group consisting of ¹¹¹In, ⁶⁷Ga, ^{99m}Tc, and ¹⁷⁷Lu.

Also provided herein are single-chain variable fragments comprising (scFv) or antigen-binding fragments thereof that bind to three extracellular loops of the transmembrane domain of ZnT8 comprising (a) a heavy chain variable region sequence of SEQ ID NO: 2 or SEQ ID NO: 19; and (b) a light chain variable region sequence of SEQ ID NO: 7. In some embodiments, the heavy chain variable region sequence comprises at least one conservative amino acid substitution within SEQ ID NO: 2 or SEQ ID NO: 19; and (b) the light chain variable region sequence comprises at least one conservative amino acid substitution within SEQ ID NO: 7.

In some embodiments, the scFv is conjugated to an imaging agent. In some embodiments, the imaging agent is a radiometal. In some embodiments, the radiometal is selected from the group consisting of ⁶⁴Cu, ⁶⁷Cu, ⁶⁸Ga, ⁶⁰Ga, ⁸⁹Zr, ⁸⁶Y, ^{94m}Tc ¹¹¹In, ⁶⁷Ga, ^{99m}Tc, and ¹⁷⁷Lu.

Also provided herein are antibodies or antigen-binding fragments thereof comprising (a) a light chain comprising SEQ ID NO: 20; and (b) a heavy chain comprising one of SEQ ID NOS: 21-24. Also provided herein are antibodies or antigen-binding fragments thereof comprising (a) a light chain comprising SEQ ID NO: 20; and (b) a heavy chain comprising one of SEQ ID NOS: 27-30.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIGS. 1A-1G show induction of anti-TMD antibodies and biochemical characterization. FIG. 1A shows a membrane-flush extracellular surface of ZnT8 (space-filling representation, left) formed by three short loops (ball-and-sticks, right) on top of a ZnT8 homodimer with bound zinc ions. The TMD is imbedded in the lipid bilayer while the CTD is extended into the cytoplasm. FIG. 1B shows sequence alignments of three extracellular loops (ECLs). FIG. 1C shows ELISA titrations of mouse sera from proteoliposome- or liposome-injected ZnT8-KO mice against either flZnT8 or CTD as indicated. FIG. 1D shows the same as in FIG. 1C expect using NOD female mice. Error bars are standard errors from 4 ZnT8-KO or 4 NOD mice, *p<0.01 (n=4). FIG. 1E shows mAb43 and mAb20 titrations against detergent-solubilized flZnT8 as indicated. FIG. 1F shows mAb43 and mAb20 titrations against CTD. FIG. 1G shows mAb43 and mAb20 titrations against ZnT8 proteoliposomes. Note, ZnT8 in proteoliposomes adopted mixed transmembrane orientations exposing either TMD or CTD to antibody binding as indicated. Solid lines are least-square fits of binding curves to a hyperbolic function with r2>0.98.

FIGS. 2A-2G show antibody bindings to ZnT8 and cell-surface markers. FIG. 2A shows IF-labeling of live EndoC-βH1 cells with mAb43 or mAb20 as indicated. The cells were counterstained with CD71 antibody and DAPI. FIG. 2B shows parallel IF-labeling of EndoC-βH1 cells after PFA-fixation and detergent permeabilization. FIG. 2C shows IF-staining of live wild-type or ZnT8-KO INS-1E cells with mAb43, Na+/K+ ATPase antibody and DAPI as indicated. FIG. 2D shows parallel IF-labeling of wild type or ZnT8-KO INS-1E cells after PFA-fixation and detergent permeabilization. FIG. 2E shows IF-labeling of live EndoC-bH1 cells with a ZnT8ecA-positive human serum, a mouse mAb43 or serum-mAb43 combinations as indicated. FIG. 2F shows quantification of cell surface (S) and intracellular (I) fluorescent intensities by mAb43 or mAb20 immunolabeling of live EndoC-bH1 cells in FIGS. 2A-2B, or mAb43 immunolabeling of either WT or ZnT8-KO INS-1E cells in FIGS. 2C-2D. The fluorescent intensities were normalized to that of mAb43 in each pair of control groups as indicated. Open circles are datapoints for individual cells. Error bars are standard errors. FIG. 2G shows quantification of cell surface IF-labeling of live EndoC-βH1 cells by a ZnT8ecA-positive human serum, mouse mAb43 or serum/mAb43 combinations as described in FIG. 2E. The fractional intensity is serum or mAb43 signal normalized to the sum of serum and mAb43 intensities for each pair of control groups as indicated.

FIGS. 3A-3D show mapping mAb43 epitope to ECLs. FIG. 3A shows sizing-HPLC chromatograms of stable protein binding complexes of ZnT8-GFP with mAb43, mAb20, or FLAG antibody as indicated. Dashed lines mark the alignment of peak positions of free or bound ZnT8-GFP as indicated. FIG. 3B shows chromatograms of stable protein binding complexes of ZnT8FLAG-GFP with mAb43, mAb20, or FLAG antibody as indicated. FIG. 3C shows immunoblotting analysis of mAb43, mAb20 and an anti-peptide ZnT8 antibody using SDS-denatured total lysate of human EndoC-βH1 cells. Arrows indicate two splice variants of endogenous ZnT8. FIG. 3D shows a side view of an electron density map of negatively stained ZnT8-Fab43 complex showing a Fab43 molecule bound to one of the two ZnT8 protomers. The oval density consists of a ZnT8 homodimer and associated detergent/lipid molecules. The cartoons are docked human ZnT8 and a Fab molecule, respectively. The dashed arrow marks the two-fold axis of a ZnT8 homodimer aligned with the minor axis of the oval.

FIGS. 4A-4F show mAb43 specificity for mouse β-cells. FIG. 4A shows mAb43 immunolabeling and diaminobenzidine detection of endogenous ZnT8 in paraffin-embedded mouse pancreas sections with mAb20 and PBS as negative controls. FIG. 4B shows IF-labeling of enzymatically dispersed islet cells from isolated mouse islets using mouse mAb43 or mouse IgG2b isotype control, followed by anti-mouse IgG-PE, anti-insulin-APC, anti-glucagon-488 and DCV. All islet cells were PFA-fixed and detergent permeabilized before immunolabeling. FIG. 4C shows mAb43 and anti-insulin co-immunolabeling of cryosections of autopsied human pancreata with DAPI counterstain. FIG. 4D shows immunolabeling and fluorescence-activated cell sorting of single cell suspension derived from enzymatically dispersed whole pancreata. Dispersed whole pancreatic cells were labeled with DCV, chimeric mAb43 or mAb20, and detected with PE-conjugated anti-human IgG as indicated. Intact cells (DCV-positive) were gated and sorted into mAb43-PE positive (R1) and negative (R0) populations. Dashed lines mark the thresholds for the DCV and mAb43-PE gate. The percentages of total intact cells within R1 and R0 gates are indicated. Data are representatives of four independent experiments. FIG. 4E shows confocal microscopy analysis of insulin and glucagon expression in different populations of mAb43-labeled cells as indicated. The sorted pancreatic cells were grown on a matrigel-coated glass surface, PFA-fixed, permeabilized and then immunolabelled by mAb43, followed by anti-insulin-APC, anti-glucagon-488 and anti-human IgG-PE as indicated. Inset, close-up view of typical β-cells within the R1 gate demonstrating co-localization of insulin and ZnT8 in the cytoplasm. FIG. 4F shows quantification of mAb43 and anti-insulin IF intensities of enriched pancreatic cells in FIG. 4E. The mAb43 or anti-insulin IF intensities are normalized to that of the R1 cell population. Open circles are datapoints for individual cells. Error bars are standard errors.

FIGS. 5A-5C show glucose-stimulated ZnT8-mAb43 uptake. FIG. 5A shows mAb43-A647 uptake in EndoC-βH1 cells at 37° C. Live cells were exposed to mAb20-A647, or mAb43-A647, in the presence of either high (20 mM) or basal (2 mM) glucose as indicated. For each image, the left panel shows A647-IF while the right panel is the merge of A647, CellMask green and DAPI signals. All scale bars are 10 μm. FIG. 5B shows cell surface mAb-A647 binding at 8° C. FIG. 5C shows imaging quantification of total A647-IF intensity in arbitrary unit (a.u.) with or without glucose stimulation (20/2 mM), at 8 or 37° C. as indicated. Open circles are datapoints for individual cells. Error bars are standard errors.

FIGS. 6A-6G show biodistributions of systemically administered antibodies in mice. FIG. 6A shows western blot analysis of SDS-solubilized tissues of different organs excised from C57BL/6 mice 1-day post-injection of chimeric mAb43 or mAb20 as indicated. Tissue proteins were loaded at 0.5 mg/lane and detected by horseradish peroxidase (HRP) chemiluminescence. FIG. 6B shows relative tissue abundance of mAb43 (black bars) or mAb20 (grey bars) in different organs. Western blot intensities were normalized to the pancreatic mAb43 signal at the same post-injection timepoints, and then averaged over four independent measurements from tissues collected at 1-, 3—, 5- and 6-days post-injection. Open circles are individual datapoints. Error bars are standard errors. FIG. 6C shows time-dependent reduction of pancreatic mAb43 or renal mAb20 as indicated. Serial dilutions of a human IgG standard were loaded onto the same gel to calibrate the mAb43 and mAb20 intensities. FIG. 6D shows quantification of pancreatic mAb43 and renal mAb20 at various post-injection timepoints as indicated. Error bars are standard errors from four independent western blot measurements. FIG. 6E shows relative tissue uptake of mAb43 in NOD (black bars) or db/db mice (white bars) as indicated. Western blot intensities were normalized to the pancreatic mAb43 signal and then averaged over four NOD or four db/db mice from tissues collected 2-days post-injection. Open circles are individual datapoints. Error bars are standard errors. FIG. 6F shows comparison of pancreatic mAb43 uptakes in three different mouse strains as indicated. The level of pancreatic mAb43 uptake is correlated with the FBG level in individual mice of different strains. FIG. 6G shows quantification of average pancreatic mAb43 uptake in different mouse stains as indicated. Open circles are western blot datapoints for individual mice, and their corresponding FBG levels are shown in the right panel. Error bars are standard errors from four mice in each mouse groups as indicated.

FIGS. 7A-7F show distribution of mAb43-mScarlet in flattened pancreas demonstrating in vivo islet-homing of mScarlet. FIG. 7A shows wholemount image of a pancreas excised from a MIP-GPF mouse receiving a mAb43-mScarlet injection at 15 mg/kg. GFP, mScarlet and bright field images were merged, and regions of interest (ROIs) used for close-up views are numbered. FIG. 7B shows close-up views of different ROIs (1, 2, 3) showing branched arterioles and colocalization of GFP and mScarlet in islet clusters. FIG. 7C shows close-up views of individual islets with overlapping GFP and mScarlet fluorescence in different ROIs (4, 5, 6). FIG. 7D shows closed-up views of individual islets with additional scattered mScarlet fluorescence in different ROIs (7, 8, 9). FIG. 7E shows mScarlet uptake in isolated mouse islets exposed to mAb43-mScarlet. FIG. 7F shows absence of mScarlet uptake in isolated mouse islets exposed to mAb20-mScarlet. Representative islet images were obtained from two independent experiments.

### DETAILED DESCRIPTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

ZnT8 is a dominant zinc transporter in j-cells with a protein expression level comparable to that of the house-keeping α-tubulin (3). This extraordinary cellular capacity of producing an active zinc transporter brings about one of the highest cellular zinc contents of 0-cells in the human body. The tissue distribution of ZnT8 is almost exclusively limited to pancreatic islets (4,5). Although ZnT8 mRNA in islets was detected in all endocrine cell types including α, β, γ, δ and ε cells, the mRNA level may be only loosely related to corresponding protein levels of ZnT8 in difference cell types (6,7). Cell sorting based on the cellular zinc content resulted in a clear separation of j-cells from other islet cells (8), suggesting that the cellular zinc content and its associated ZnT8 protein level are specific biomarkers for β-cells. The subcellular distribution of ZnT8 is in a dynamic equilibrium among the cell surface membrane, insulin secretory granule and endoplasmic reticulum where ZnT8 functions as a zinc-sequestering transporter (3,9,10). The enriched zinc ions are required for proinsulin processing and crystalline packaging of zinc-insulin hexamers (9-13). As a result, ZnT8 subcellular distribution is tightly coupled with insulin processing, storage and secretion (14). Glucose stimulated insulin secretion promotes ZnT8 trafficking to the cell surface (15), making it a major cell-surface antigenic target for autoantibodies in patients with type-1 diabetes (16). Likewise, the surfaced ZnT8 could potentially act as a functional biomarker for mAb-based immunodetection.

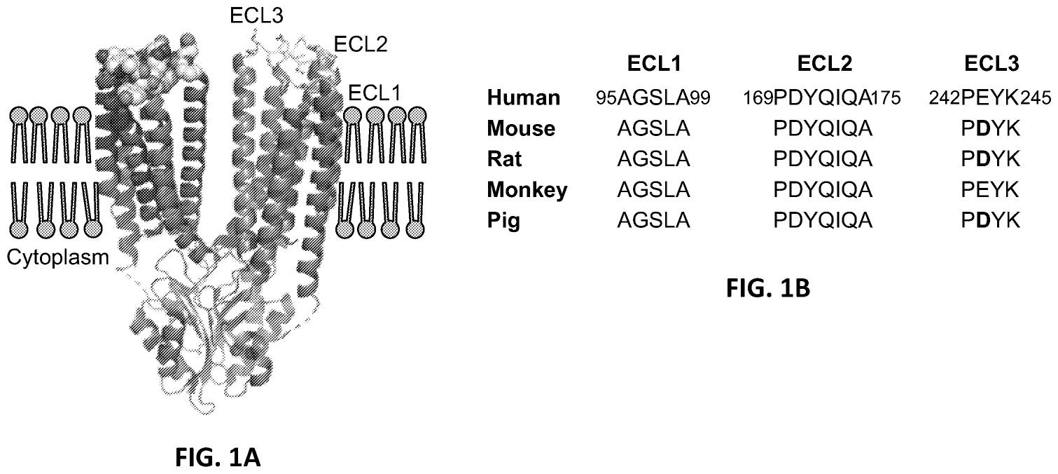

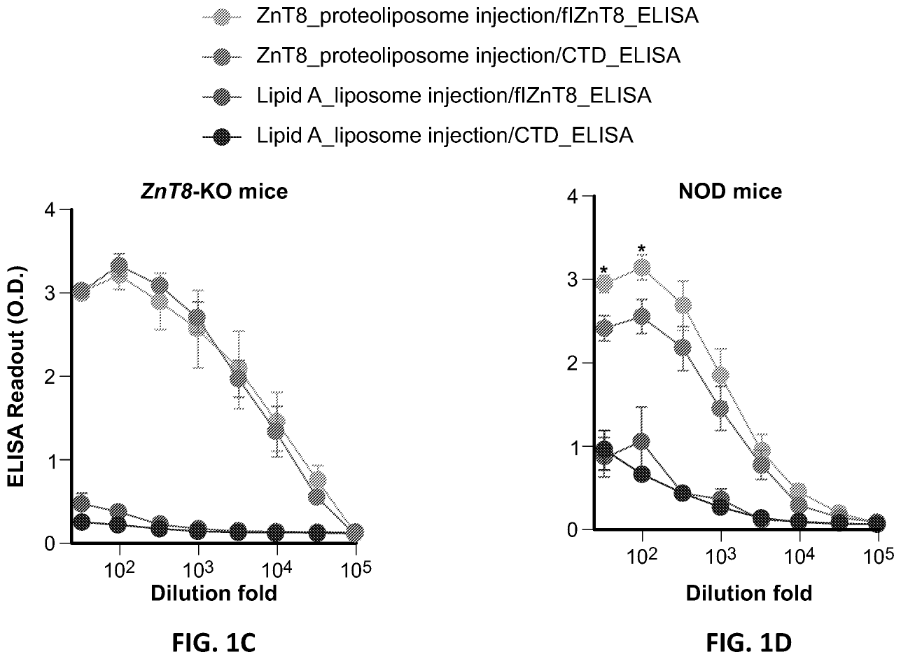

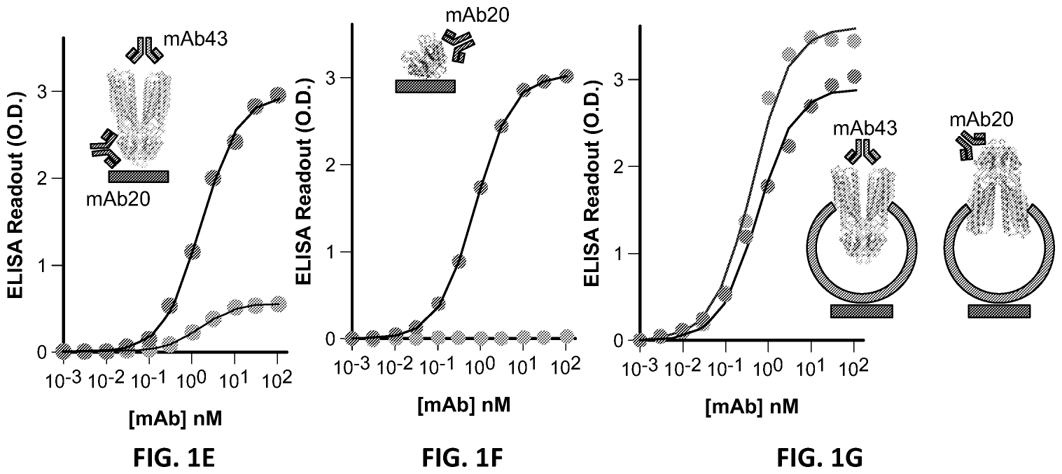

An earlier ZnT8 mAb to a linear peptide derived from an extracellular loop of ZnT8 yielded modest binding affinity (108 nM) and low specificity (17). In vivo β-cell imaging and targeting require the development of high-affinity mAbs with exquisite conformation specificity to multiple extracellular loops arranged in spatial configurations. ZnT8 is a two-modular protein consisting of a compact transmembrane domain (TMD) and a cytosolic C-terminal domain (CTD). The TMD lacks an ectodomain while its extracellular surface is membrane-flush and formed by three short extracellular loops (ECL1-3) (FIG. 1A). In addition to limited epitope availability, these loops are poorly antigenic because they are quasi-invariant between mouse and human ZnT8 with the exception of a highly conserved E-to-D substitution in ECL3 (FIG. 1B). As described herein, a mouse immunization strategy has been established to enhance immunogenicity of extracellular epitopes in natively-folded ZnT8, identified mAb43 to extracellular loops with conformation-specificity, and demonstrated the utility of mAb43 for β-cell purification and targeted delivery of imaging-probes.

### I. Definitions

The term “antibody” means an immunoglobulin molecule that recognizes and specifically binds to a target, such as a protein (e.g., the ZNT8, a subunit thereof, or the receptor complex), polypeptide, peptide, carbohydrate, polynucleotide, lipid, or combinations of the foregoing through at least one antigen recognition site within the variable region of the immunoglobulin molecule. A typical antibody comprises at least two heavy (HC) chains and two light (LC) chains interconnected by disulfide bonds. Each heavy chain is comprised of a “heavy chain variable region” or “heavy chain variable domain” (abbreviated herein as VH) and a heavy chain constant region. The heavy chain constant region is comprised of three domains, CH1, CH2, and CH3. Each light chain is comprised of a “light chain variable region” or “light chain variable domain” (abbreviated herein as VL) and alight chain constant region. The light chain constant region is comprised of one domain, CI. The VH and VL regions can be further subdivided into regions of hypervariability, termed Complementarity Determining Regions (CDR), interspersed with regions that are more conserved, termed framework regions (FRs). Each VH and VL region is composed of three CDRs and four FRs, arranged from amino-terminus to carboxy-terminus in the following order: FRI, CDRI, FR2, CDR2, FR3, CDR3, FR4. The variable regions of the heavy and light chains contain a binding domain that interacts with an antigen. As used herein, the term “antibody” encompasses intact poly clonal antibodies, intact monoclonal antibodies, antibody fragments (such as Fab, Fab′, F(ab′)2, Fd, Facb, and Fv fragments), single chain Fv (scFv), minibodies (e.g., sc(Fv)2, diabody), multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. Thus, the term “antibody” includes whole antibodies and any antigen-binding fragment or single chains thereof. Antibodies can be naked or conjugated to other molecules such as toxins, radioisotopes, small molecule drugs, polypeptides, etc.

The term “isolated antibody” refers to an antibody that has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In some embodiments, the antibody is purified (1) to greater than 95% by weight of antibody as determined by, for example, the Lowry method, and including more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or silver stain. An isolated antibody includes the antibody in situ within recombinant cells since at least one component of the antibody's natural environment will not be present. Ordinarily, however, isolated antibody will be prepared by at least one purification step.

The term “humanized” immunoglobulin refers to an immunoglobulin comprising a human framework region and one or more CDRs from a non-human (usually a mouse or rat) immunoglobulin. The non-human immunoglobulin providing the CDRs is called the “donor” and the human immunoglobulin providing the framework is called the “acceptor.” Constant regions need not be present, but if they are, they must be substantially identical to human immunoglobulin constant regions, i.e., at least about 85-90%, preferably about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. For example, a humanized antibody would not encompass a typical chimeric antibody as defined above, e.g., because the entire variable region of a chimeric antibody is non-human.

The term “antigen binding fragment” refers to a portion of an intact antibody and refers to the antigenic determining variable regions of an intact antibody. Itis known in the art that the antigen binding function of an antibody can be performed by fragments of a full-length antibody. Examples of antigen-binding antibody fragments include, but are not limited to Fab, Fab′, F(ab′)2, Facb, Fd, and Fv fragments, linear antibodies, single chain antibodies, and multi-specific antibodies formed from antibody fragments. In some instances, antibody fragments may be prepared by proteolytic digestion of intact or whole antibodies. For example, antibody fragments can be obtained by treating the whole antibody with an enzyme such as papain, pepsin, or plasmin. Papain digestion of whole antibodies produces F(ab)2 or Fab fragments; pepsin digestion of whole antibodies yields F(ab′)2 or Fab′; and plasmin digestion of whole antibodies yields Facb fragments.

The term “Fab” refers to an antibody fragment that is essentially equivalent to that obtained by digestion of immunoglobulin (typically IgG) with the enzyme papain. The heavy chain segment of the Fab fragment is the Fd piece. Such fragments can be enzymatically or chemically produced by fragmentation of an intact antibody, recombinantly produced from a gene encoding the partial antibody sequence, or it can be wholly or partially synthetically produced. The term “F(ab′)2” refers to an antibody fragment that is essentially equivalent to a fragment obtained by digestion of an immunoglobulin (typically IgG) with the enzyme pepsin at pH 4.0-4.5. Such fragments can be enzymatically or chemically produced by fragmentation of an intact antibody, recombinantly produced from a gene encoding the partial antibody sequence, or it can be wholly or partially synthetically produced. The term “Fv” refers to an antibody fragment that consists of one NH and one N domain held together by noncovalent interactions.

The terms “ZNT8 antibody,” “anti-ZNT8 antibody,” “anti-ZNT8,” “antibody that binds to ZNT8” and any grammatical variations thereof refer to an antibody that is capable of specifically binding to ZNT8 with sufficient affinity such that the antibody is useful as a therapeutic agent or diagnostic reagent in targeting ZNT8. The extent of binding of an anti-ZNT8 antibody disclosed herein to an unrelated, non-ZNT8 protein is less than about 10% of the binding of the antibody to ZNT8 as measured, e.g., by a radioimmunoassay (RIA), BIACORE™ (using recombinant ZNT8 as the analyte and antibody as the ligand, or vice versa), or other binding assays known in the art. In certain embodiments, an antibody that binds to ZNT8 has a dissociation constant (KD) of <1 μM, <100 nM, <50 nM, <10 nM, or <1 nM.

The term “% identical” between two polypeptide (or polynucleotide) sequences refers to the number of identical matched positions shared by the sequences over a comparison window, considering additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence. The percentage of sequence identity is calculated by determining the number of positions at which the identical amino acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software both for online use and for download. Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site. Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa. In certain embodiments, the percentage identity “X” of a first amino acid sequence to a second sequence amino acid is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence. One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2 (ClustalX is a version of the ClustalW2 program ported to the Windows environment). Another suitable program is MUSCLE. ClustalW2 and MUSCLE are alternatively available, e.g., from the European Bioinformatics Institute (EBI).

The term “therapeutic agent” refers to any biological or chemical agent used in the treatment of a disease or disorder. Therapeutic agents include any suitable biologically active chemical compounds, biologically derived components such as cells, peptides, antibodies, and polynucleotides, and radiochemical therapeutic agents such as radioisotopes. In some embodiments, the therapeutic agent comprises a chemotherapeutic agent or an analgesic.

As used herein, the terms “treatment,” “treating,” “treat” and the like, refer to obtaining a desired pharmacologic and/or physiologic effect. The terms are also used in the context of the administration of a “therapeutically effective amount” of an agent, e.g., an anti-ZnT8 antibody. The effect may be prophylactic in terms of completely or partially preventing a particular outcome, disease or symptom thereof and/or may be therapeutic in terms of a partial or complete cure for a disease and/or adverse effect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease. In particular embodiments, the term is used in the context of preventing or treating any ZnT8-mediated disease including diabetes.

### II. Anti-ZnT8 Antibodies

The antibodies or antigen-binding fragment thereof of this disclosure specifically bind to ZNT8. In specific embodiments, these antibodies or antigen-binding fragments specifically bind to human ZNT8. In particular embodiments, these antibodies or antigen-binding fragments specifically bind to the transmembrane domain of ZNT8. “Specifically binds” as used herein means that the antibody or antigen-binding fragment preferentially binds ZNT8 (e.g., human ZNT8, mouse ZNT8) over other proteins. In certain instances, the anti-ZNT8 antibodies of the disclosure have a higher affinity for ZNT8 than for other proteins. Anti-ZNT8 antibodies that specifically bind ZNT8 may have a binding affinity for human ZNT8 of less than or equal to 1×10⁻⁷ M, less than or equal to 2×10⁻⁷ M, less than or equal to 3×10⁻⁷ M, less than or equal to 4×10⁻⁷ M, less than or equal to 5×10⁻⁷ M, less than or equal to 6×10⁻⁷ M, less than or equal to 7×10⁻⁷ M, less than or equal to 8×10⁻⁷ M, less than or equal to 9×10⁻⁷ M, less than or equal to 1×10⁻⁸ M, less than or equal to 2×10⁻⁸ M, less than or equal to 3×10⁻⁸ M, less than or equal to 4×10⁻⁸ M, less than or equal to 5×10⁻⁸ M, less than or equal to 6×10⁻⁸ M, less than or equal to 7×10⁻⁸ M, less than or equal to 8×10⁻⁸ M, less than or equal to 9×10⁻⁸ M, less than or equal to 1×10⁻⁹ M, less than or equal to 2×10⁻⁹ M, less than or equal to 3×10⁻⁹ M, less than or equal to 4×10⁻⁹ M, less than or equal to 5×10⁻⁹ M, less than or equal to 6×10⁻⁹ M, less than or equal to 7×10⁻⁹ M, less than or equal to 8×10⁻⁹ M, less than or equal to 9×10⁻⁹ M, less than or equal to 1×10⁻¹⁰ M, less than or equal to 2×10⁻¹⁰ M, less than or equal to 3×10⁻¹⁰ M, less than or equal to 4×10⁻¹⁰ M, less than or equal to 5×10⁻¹⁰ M, less than or equal to 6×10⁻¹⁰ M, less than or equal to 7×10⁻¹⁰ M, less than or equal to 8×10⁻¹⁰ M, less than or equal to 9×10⁻¹⁰ M, less than or equal to 1×10⁻¹¹ M, less than or equal to 2×10⁻¹¹ M, less than or equal to 3×10⁻¹¹ M, less than or equal to 4×10⁻¹¹ M, less than or equal to 5×10⁻¹¹ M, less than or equal to 6×10⁻¹¹ M, less than or equal to 7×10⁻¹¹ M, less than or equal to 8×10⁻¹¹ M, less than or equal to 9×10⁻¹¹ M, less than or equal to 1×10⁻¹² M, less than or equal to 2×10⁻¹² M, less than or equal to 3×10⁻¹² M, less than or equal to 4×10⁻¹² M, less than or equal to 5×10⁻¹² M, less than or equal to 6×10⁻¹² M, less than or equal to 7×10⁻¹² M, less than or equal to 8×10⁻¹² M, or less than or equal to 9×10⁻¹² M. Methods of measuring the binding affinity of an antibody are well known in the art and include Surface Plasmon Resonance (SPR) (Morton and Myszka “Kinetic analysis of macromolecular interactions using surface plasmon resonance biosensors” Methods in Enzymology (1998) 295, 268-294), Bio-Layer Interferometry, (Abdiche et al “Determining Kinetics and Affinities of Protein Interactions Using a Parallel Real-time Label-free Biosensor, the Octet” Analytical Biochemistry (2008) 377, 209-217), Kinetic Exclusion Assay (KinExA) (Darling and Brault “Kinetic exclusion assay technology: characterization of molecular interactions” Assay and Drug Dev Tech (2004) 2, 647-657), isothermal calorimetry (Pierce et al “Isothermal Titration Calorimetry of Protein-Protein Interactions” Methods (1999) 19, 213-221) and analytical ultracentrifugation (Lebowitz et al “Modem analytical ultracentrifugation in protein science: A tutorial review” Protein Science (2002), 11:2067-2079).

In one aspect, provided herein is an anti-ZnT8 antibody or antigen-binding fragment thereof comprising a heavy chain variable region and a light chain variable region, wherein the heavy chain variable region comprises (i) CDR-H1 comprising the amino acid sequence of SEQ ID NO:3, (ii) CDR-H2 comprising the amino acid sequence of SEQ ID NO:4, and (iii) CDR-H3 comprising the amino acid sequence of SEQ ID NO:5; and/or wherein the light chain variable region comprises (i) CDR-L1 comprising the amino acid sequence of SEQ ID NO:8, (ii) CDR-L2 comprising the amino acid sequence of SEQ ID NO:9, and (iii) CDR-L3 comprising the amino acid sequence of SEQ ID NO: 10, wherein the CDRs of the anti-ZnT8 antibody are defined by the Kabat numbering scheme.

In one aspect, provided herein is an anti-ZnT8 antibody or antigen-binding fragment thereof comprising a heavy chain variable domain comprising the amino acid sequence of SEQ ID NO:2 and comprising a light chain variable domain comprising the amino acid sequence of SEQ ID NO:7.

In some embodiments, provided herein is an anti-ZnT8 antibody or antigen-binding fragment thereof comprising a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:2 or SEQ ID NO:19. In certain embodiments, a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:2 or SEQ ID NO:19 contains substitutions (e.g., conservative substitutions), insertions, or deletions relative to the reference sequence and retains the ability to bind to a ZnT8 (e.g., human ZnT8). In certain embodiments, a total of 1 to 10 amino acids have been substituted, inserted and/or deleted in SEQ ID NO:2 or SEQ ID NO:19. In certain embodiments, substitutions, insertions, or deletions (e.g., 1, 2, 3, 4, or 5 amino acids) occur in regions outside the CDRs (i.e., in the FRs). In some embodiments, the anti-ZnT8 antibody comprises a heavy chain variable domain sequence of SEQ ID NO:2 or SEQ ID NO:19 including post-translational modifications of that sequence. In certain embodiments, a heavy chain variable domain sequence contains one point mutation relative to SEQ ID NO:2 or SEQ ID NO:19. In further embodiments, the one point mutation is located in a CDR region.

In some embodiments, provided herein is an anti-ZnT8 antibody or antigen-binding fragment thereof comprising a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:7. In certain embodiments, a light chain variable domain comprising an amino acid sequence having at least 85%, 86%, 87%, 88%, 89%, 90%, 91%, 92%, 93%, 94%, 95%, 96%, 97%, 98% or 99% sequence identity to the amino acid sequence of SEQ ID NO:7 contains substitutions (e.g., conservative substitutions), insertions, or deletions relative to the reference sequence and retains the ability to bind to a ZnT8 (e.g., human ZnT8). In certain embodiments, a total of 1 to 10 amino acids have been substituted, inserted and/or deleted in SEQ ID NO:7. In certain embodiments, substitutions, insertions, or deletions (e.g., 1, 2, 3, 4, or 5 amino acids) occur in regions outside the CDRs (i.e., in the FRs). In some embodiments, the anti-ZnT8 antibody comprises a light chain variable domain sequence of SEQ ID NO:7 including post-translational modifications of that sequence. In certain embodiments, a light chain variable domain sequence contains at least one point mutation relative to SEQ ID NO:7. In further embodiments, the one point nutation is located in a CDR region.

Thus, in particular embodiments, the sequences can comprise at least one conservative substitution. It is understood that the phrase “at least one” is synonymous with “one or more” and includes values such as at least 1, at least 2, at least 3, at least 4, at least 5, at least 6, at least 7, at least 8, at least 9, at least 10, at least 11, at least 12, at least 13, at least 14, at least 15 . . . at least “N” wherein “N” equals the total number of amino acids in the particular sequence (and therefore, 1 or more, 2 or more, 3 or more, etc.).

The sequences can also comprise up to 1, up to 2, up to 3, up to 4, up to 5, up to 6, up to 7, up to 8, up to 9, up to 10, up to 11, up to 12, up to 13, up to 14, up to 15 . . . up to “N” wherein “N” equals the total number of amino acids in the particular sequence. Alternatively, a particular sequence can comprise a substitution at 10 or fewer, 9 or fewer, 8 or fewer, 7 or fewer, 6 or fewer, 5 or fewer, 4 or fewer, 3 or fewer, 2 or fewer, etc. amino acid positions.

In specific embodiments, the present invention provides an isolated antibody or antibody-binding fragment thereof that specifically binds to ZnT8, wherein the antibody or antibody-binding fragment comprises heavy chain complementarity determining regions (CDRs) 1, 2 and 3, wherein the heavy chain CDR1 comprises an amino acid sequence as set forth in SEQ ID NO:3, or the amino acid sequence as set forth in SEQ ID NO:3 with a substitution at three or fewer amino acid positions, the heavy chain CDR2 comprising an amino acid set forth in SEQ ID NO:4, or the amino acid set forth in SEQ ID NO:4 with a substitution at seven or fewer amino acid positions, and the heavy chain CDR3 comprising an amino acid sequence as set forth in SEQ ID NO:5, or the amino acid sequence as set forth in SEQ ID NO:5 with a substitution at four or fewer amino acid positions.

In further embodiments, the isolated antibody or antigen-binding fragment further comprises light chain CDRs 1, 2 and 3, wherein the light chain CDR1 comprises an amino acid sequence as set forth in SEQ ID NO:8, or the amino acid sequence as set forth in SEQ ID NO:8 with a substitution at six or fewer amino acid positions, the light chain CDR2 comprising an amino acid sequence as set forth in SEQ ID NO:9, or the amino acid sequence as set forth in SEQ ID NO:9 with a substitution at four or fewer amino acid positions, and the light chain CDR3 comprising an amino acid sequence as set forth in SEQ ID NO:10, or the amino acid sequence as set forth in SEQ ID NO:10 with a substitution at five or fewer amino acid positions.

In some embodiments, the anti-ZnT8 antibody or the anti-ZnT8 antibody of the anti-ZnT8 antibody-drug conjugate is a monoclonal antibody.

There are five classes of immunoglobulins: IgA, LgD, IgE, IgG and IgM, having heavy chains designated α, δ, ε, γ, and μ, respectively. The γ and μ classes are further divided into subclasses e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 41-7) any of which are suitable for use in some of the embodiments herein. Common allotypic variants in human populations are those designated by the letters a, f, n, z or combinations thereof. In any of the embodiments herein, the antibody may comprise a heavy chain Fc region comprising a human IgG Fc region. In further embodiments, the human IgG Fc region comprises a human IgG4.

The antibodies may also include derivatives that are modified, i.e., by the covalent attachment of any type of molecule to the antibody such that covalent attachment does not prevent the antibody from binding to ZnT8 or from exerting a cytostatic or cytotoxic effect on cells. For example, but not by way of limitation, the antibody derivatives include antibodies that have been modified, e.g., by glycosylation, acetylation, PEGylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

### Antibody Fragments

The present disclosure encompasses the antibody fragments or domains described herein that retains the ability to specifically bind to ZNT8 (e.g., human ZNT8 including, but not limited to, the transmembrane domain of ZNT8). Antibody fragments include, e.g., Fab, Fab′, F(ab′)2, Facb, and Fv. These fragments may be humanized or fully human. Antibody fragments may be prepared by proteolytic digestion of intact antibodies. For example, antibody fragments can be obtained by treating the whole antibody with an enzyme such as papain, pepsin, or plasmin. Papain digestion of whole antibodies produces F(ab)2 or Fab fragments; pepsin digestion of whole antibodies yields F(ab′)2 or Fab′; and plasmin digestion of whole antibodies yields Facb fragments.

Alternatively, antibody fragments can be produced recombinantly. For example, nucleic acids encoding the antibody fragments of interest can be constructed, introduced into an expression vector, and expressed in suitable host cells. See, e.g., Co, M. S. et al., J Immunol., 152:2968-2976 (1994); Better, M. and Horwitz, A. H., Methods in Enzymology, 178:476-496 (1989); Pluckthun, A and Skerra, A, Methods in Enzymology, 178:476-496 (1989); Lamoyi, E., Methods in Enzymology, 121:652-663 (1989); Rousseaux, J. et al., Methods in Enzymology, (1989) 121:663-669 (1989); and Bird, R E. et al., TIBTECH, 9:132-137 (1991)). Antibody fragments can be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab)2 fragments (Carter et al., Bio/Technology, 10:163-167 (1992)). According to another approach, F(ab′)2 fragments can be isolated directly from recombinant host cell culture. Fab and F(ab′) 2 fragment with increased in vivo half-life comprising a salvage receptor binding epitope residues are described in U.S. Pat. No. 5,869,046.

### Minibodies

Also encompassed are minibodies of the antibodies described herein. Minibodies of anti-ZNT8 antibodies include diabodies, single chain (scFv), and single-chain (Fv)2 (sc(Fv)2).

A “diabody” is a bivalent minibody constructed by gene fusion (see, e.g., Holliger, P. et al., Proc. Natl. Acad. Sci. U S. A., 90:6444-6448 (1993); EP 404,097; WO 93/11161). Diabodies are dimers composed of two polypeptide chains. The VL and VH domain of each polypeptide chain of the diabody are bound by linkers. The number of amino acid residues that constitute a linker can be between 2 to 12 residues (e.g., 3-10 residues or five or about five residues). The linkers of the polypeptides in a diabody are typically too short to allow the VL and VH to bind to each other. Thus, the VL and VH encoded in the same polypeptide chain cannot form a single-chain variable region fragment, but instead form a dimer with a different single-chain variable region fragment. As a result, a diabody has two antigen-binding sites.

An scFv is a single-chain polypeptide antibody obtained by linking the VH and VL with a linker (see e.g., Huston et al., Proc. Natl. Acad. Sci. U S. A., 85:5879-5883 (1988); and Pluckthun, “The Pharmacology of Monoclonal Antibodies” Vol. 113, Ed Resenburg and Moore, Springer Verlag, New York, pp. 269-315, (1994)). Each variable domain (or a portion thereof) is derived from the same or different antibodies. Single chain Fv molecules preferably comprise an scFv linker interposed between the VH domain and the VL domain. Exemplary scFv molecules are known in the art and are described, for example, in U.S. Pat. No. 5,892,019; Ho et al, Gene, 77:51 (1989); Bird et al., Science, 242:423 (1988); Pantoliano et al, Biochemistry, 30: 101 17 (1991); Milenic et al, Cancer Research, 51:6363 (1991); Takkinen et al, Protein Engineering, 4:837 (1991).

The term “scFv linker” as used herein refers to a moiety interposed between the VL and VH domains of the scFv. The scFv linkers preferably maintain the scFv molecule in an antigen-binding conformation. In some embodiments, an scFv linker comprises or consists of an scFv linker peptide. In certain embodiments, an scFv linker peptide comprises or consists of a Gly-Ser peptide linker. In some embodiments, an scFv linker comprises a disulfide bond.

The order of VHs and VLs to be linked is not particularly limited, and they may be arranged in any order. Examples of arrangements include: [VH] linker [VL]; or [VL] linker [VH]. The H chain V region and L chain V region in an scFv may be derived from any anti-ZNT8 antibody or antigen-binding fragment thereof described herein.

An sc(Fv)2 is a minibody in which two VHs and two VLs are linked by a linker to form a single chain (Hudson, et al., J Immunol. Methods, (1999) 231: 177-189 (1999)). An sc(Fv)2 can be prepared, for example, by connecting scFvs with a linker. The sc(Fv)2 of the present invention include antibodies preferably in which two VHs and two VLs are arranged in the order of: VH, VL, VH, and VL ([VH] linker [VL] linker [VH] linker [VL]), beginning from the N terminus of a single-chain polypeptide; however, the order of the two VHs and two VLs is not limited to the above arrangement, and they may be arranged in any order. Examples of arrangements are listed below:
[VL] linker [VH] linker [VH] linker [VL]
[VH] linker [VL] linker [VL] linker [VH]
[VH] linker [VH] linker [VL] linker [VL]
[VL] linker [VL] linker [VH] linker [VH]
[VL] linker [VH] linker [VL] linker [VH]

Normally, three linkers are required when four antibody variable regions are linked; the linkers used may be identical or different. There is no particular limitation on the linkers that link the VH and VL regions of the minibodies. In some embodiments, the linker is a peptide linker. Any arbitrary single-chain peptide comprising about 3 to 25 residues (e.g., 5, 6, 7, 8, 9, 10, 11, 12, 13, 14, 15, 16, 17, 18) can be used as a linker.

In some embodiments, the linker is a synthetic compound linker (chemical cross-linking agent). Examples of cross-linking agents that are available on the market include N-hydroxysuccinimide (NHS), disuccinimidylsuberate (DSS), bis(sulfosuccinimidyl)suberate (BS3), dithiobis(succinimidy Ipropionate) (DSP), dithiobis(sulfosuccinimidy Ipropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis[2-(succinimidooxycarbonyloxy)ethyl]sulfone (BSOCOES), and bis[2-(sulfosuccinimidooxycarbonyloxy)ethyl]sulfone (sulfo-BSOCOES).

The amino acid sequence of the VH or VL in the antibody fragments or minibodies may include modifications such as substitutions, deletions, additions, and/or insertions. For example, the modification may be in one or more of the CDRs of the anti-ZNT8 antibodies described herein. In certain embodiments, the modification involves one, two, or three amino acid substitutions in one, two, or three CDRs of the VH and/or one, two, or three CDRs of the VL domain of the anti-ZNT8 minibody. Such substitutions are made to improve the binding and/or functional activity of the anti-ZNT8 minibody. In some embodiments, one, two, or three amino acids of one or more of the six CDRs of the anti-ZNT8 antibody or antigen-binding fragment thereof may be deleted or added as long as there is ZNT8 binding and/or functional activity when VH and VL are associated.

### VHH

VHH also known as nanobodies are derived from the antigen-binding variable heavy chain regions (VHHs) of heavy chain antibodies found in camels and llamas, which lack light chains. The present disclosure encompasses VHHs that specifically bind ZNT8.

### Variable Domain of New Antigen Receptors (VNARs)

A VNAR is a variable domain of anew antigen receptor (IgNAR). IgNARs exist in the sera of sharks as a covalently linked heavy chain homodimer. It exists as a soluble and receptor bound form consisting of a variable domain (VNAR) with differing numbers of constant domains. The VNAR is composed of a CDR1 and CDR3 and in lieu of a CDR2 has HV2 and HV4 domains (see, e.g., Barelle and Porter, Antibodies, 4:240-258 (2015)). The present disclosure encompasses VNARs that specifically bind ZNT8.

### Constant Regions

Antibodies of this disclosure can be whole antibodies or single chain Fc (scFc) and can comprise any constant region known in the art. The light chain constant region can be, for example, a kappa- or lambda-type light chain constant region, e.g., a human kappa or human lambda light chain constant region. The heavy chain constant region can be, e.g., an alpha-, delta-, epsilon-, gamma-, or mu-type heavy chain constant region, e.g., a human alpha-human delta-, human epsilon-, human gamma-, or human mu-type heavy chain constant region. In certain instances, the anti-ZNT8 antibody is an IgA antibody, an IgD antibody, an IgE antibody, an IgG1 antibody, an IgG2 antibody, an IgG3 antibody, an IgG4 antibody, or an IgM antibody.

In some embodiments, the light or heavy chain constant region is a fragment, derivative, variant, or mutein of a naturally occurring constant region. In some embodiments, the variable heavy chain of the anti-ZNT8 antibodies described herein is linked to a heavy chain constant region comprising a CH1 domain and a hinge region. In some embodiments, the variable heavy chain is linked to a heavy chain constant region comprising a CH2 domain. In some embodiments, the variable heavy chain is linked to a heavy chain constant region comprising a CH3 domain. In some embodiments, the variable heavy chain is linked to a heavy chain constant region comprising a CH2 and CH3 domain. In some embodiments, the variable heavy chain is linked to a heavy chain constant region comprising a hinge region, a CH2 and a CH3 domain. The CH1, hinge region, CH2, and/or CH3 can be from an IgG antibody (e.g., IgG1, IgG4). In certain embodiments, the variable heavy chain of an anti-ZNT8 antibody described herein is linked to a heavy chain constant region comprising a CHI domain, hinge region, and CH2 domain from IgG4 and a CH3 domain from IgG1. In certain embodiments such a chimeric antibody may contain one or more additional mutations in the heavy chain constant region that increase the stability of the chimeric antibody. In certain embodiments, the heavy chain constant region includes substitutions that modify the properties of the antibody.

In certain embodiments, an anti-ZNT8 antibody of this disclosure is an IgG isotype antibody. In some embodiments, the antibody is IgG1. In another embodiment, the antibody is IgG2. In yet another embodiment, the antibody is IgG4. In some instances, the IgG4 antibody has one or more mutations that reduce or prevent it adopting a functionally monovalent format. For example, the hinge region of IgG4 can be mutated to make it identical in amino acid sequence to the hinge region of human IgG1 (mutation of a serine in human IgG4 hinge to a proline). In some embodiments, the antibody has a chimeric heavy chain constant region (e.g., having the CH1, hinge, and CH2 regions of IgG4 and CH3 region of IgG1).

### Bispecific Antibodies

In certain embodiments, an anti-ZNT8 antibody of this disclosure is a bispecific antibody. Bispecific antibodies are antibodies that have binding specificities for at least two different epitopes. Exemplary bispecific antibodies may bind to two different epitopes of the ZNT8 protein. Other such antibodies may combine a ZNT8 binding site with a binding site for another protein. Bispecific antibodies can be prepared as full length antibodies or low molecular weight forms thereof (e.g., F(ab′) 2 bispecific antibodies, sc(Fv)2 bispecific antibodies, diabody bispecific antibodies).

Traditional production of full length bispecific antibodies is based on the co-expression of two immunoglobulin heavy chain-light chain pairs, where the two chains have different specificities (Millstein et al., Nature, 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the CH3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

The “diabody” technology provides an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites.

### Conjugated Antibodies

The antibodies or antigen-binding fragments disclosed herein may be conjugated to various molecules including macromolecular substances such as polymers (e.g., polyethylene glycol (PEG), polyethylenimine (PEI) modified with PEG (PEI-PEG), polyglutamic acid (PGA) (N-(2-Hydroxypropyl) methacrylamide (HPMA) copolymers), human serum albumin or a fragment thereof, radioactive materials (e.g., ⁹⁰Y, ¹³¹I), fluorescent substances, luminescent substances, haptens, enzymes, metal chelates, and drugs.

In certain embodiments, an anti-ZNT8 antibody or antigen-binding fragment thereof is modified with a moiety that improves its stabilization and/or retention in circulation, e.g., in blood, serum, or other tissues, e.g., by at least 1.5, 2, 5, 10, 15, 20, 25, 30, 40, or 50 fold. For example, the anti-ZNT8 antibody or antigen-binding fragment thereof can be associated with (e.g., conjugated to) a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used. For example, the anti-ZNT8 antibody or antigen-binding fragment thereof can be conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides.

The above-described conjugated antibodies or fragments can be prepared by performing chemical modifications on the antibodies or the lower molecular weight forms thereof described herein. Methods for modifying antibodies are well known in the art.

### III. Characterization of Antibodies

The ZNT8 binding properties of the antibodies described herein may be measured by any standard method, e.g., one or more of the following methods: OCTET®, Surface Plasmon Resonance (SPR), BIACORE™ analysis, Enzyme Linked Immunosorbent Assay (ELISA), EIA (enzyme immunoassay), RIA (radioimmunoassay), and Fluorescence Resonance Energy Transfer (FRET).

The binding interaction of a protein of interest (an anti-ZNT8 antibody or functional fragment thereof) and a target (e.g., ZNT8) can be analyzed using the OCTET® systems. In this method, one of several variations of instruments (e.g., OCTET® QKe and QK), made by the ForteBio company are used to determine protein interactions, binding specificity, and epitope mapping. The OCTET® systems provide an easy way to monitor real-time binding by measuring the changes in polarized light that travels down a custom tip and then back to a sensor.

The binding interaction of a protein of interest (an anti-ZNT8 antibody or functional fragment thereof) and a target (e.g., ZNT8) can be analyzed using Surface Plasmon Resonance (SPR). SPR or Biomolecular Interaction Analysis (BIA) detects biospecific interactions in real time, without labeling any of the interactants.

Changes in the mass at the binding surface (indicative of a binding event) of the BIA chip result in alterations of the refractive index of light near the surface (the optical phenomenon of surface plasmon resonance (SPR)). The changes in the refractivity generate a detectable signal, which is measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem 63:2338-2345; Szabo et al. (1995) Curr. Opin. Struct. Biol. 5:699-705 and on-line resources provide by BIAcore International AB (Uppsala, Sweden). Information from SPR can be used to provide an accurate and quantitative measure of the equilibrium dissociation constant (Kd), and kinetic parameters, including Kon and Koff, for the binding of a biomolecule to a target.

Epitopes can also be directly mapped by assessing the ability of different anti-ZNT8 antibodies or functional fragments thereof to compete with each other for binding to human ZNT8 using BIACORE chromatographic techniques (Pharmacia BIAtechnology Handbook, “Epitope Mapping”, Section 6.3.2, (May 1994); see also Johne et al. (1993) J. Immunol. Methods, 160:191-198).

When employing an enzyme immunoassay, a sample containing an antibody, for example, a culture supernatant of antibody-producing cells or a purified antibody is added to an antigen-coated plate. A secondary antibody labeled with an enzyme such as alkaline phosphatase is added, the plate is incubated, and after washing, an enzyme substrate such as p-nitrophenylphosphate is added, and the absorbance is measured to evaluate the antigen binding activity.

Additional general guidance for evaluating antibodies, e.g., Western blots and immunoprecipitation assays, can be found in Antibodies: A Laboratory Manual, ed. by Harlow and Lane, Cold Spring Harbor press (1988)).

### IV. Affinity Maturation

In some embodiments, an anti-ZNT8 antibody or antigen-binding fragment thereof is modified, e.g., by mutagenesis, to provide a pool of modified antibodies. The modified antibodies are then evaluated to identify one or more antibodies having altered functional properties (e.g., improved binding, improved stability, reduced antigenicity, or increased stability in vivo). In one implementation, display library technology is used to select or screen the pool of modified antibodies. Higher affinity antibodies are then identified from the second library, e.g., by using higher stringency or more competitive binding and washing conditions. Other screening techniques can also be used. Methods of effecting affinity maturation include random mutagenesis (e.g., Fukuda et al., Nucleic Acids Res., 34:e127 (2006); targeted mutagenesis (e.g., Rajpal et al., Proc. Natl. Acad. Sci. USA, 102:8466-71 (2005); shuffling approaches (e.g., Jermutus et al., Proc. Natl. Acad. Sci. USA, 98:75-80 (2001); and in silica approaches (e.g., Lippow et al., Nat. Biotechnol., 25: 1171-6 (2005).

In some embodiments, the mutagenesis is targeted to regions known or likely to be at the binding interface. If, for example, the identified binding proteins are antibodies, then mutagenesis can be directed to the CDR regions of the heavy or light chains as described herein. Further, mutagenesis can be directed to framework regions near or adjacent to the CDRs, e.g., framework regions, particularly within 10, 5, or 3 amino acids of a CDR junction. In the case of antibodies, mutagenesis can also be limited to one or a few of the CDRs, e.g., to make step-wise improvements.

In some embodiments, mutagenesis is used to make an antibody more similar to one or more germline sequences. One exemplary germlining method can include: identifying one or more germline sequences that are similar (e.g., most similar in a particular database) to the sequence of the isolated antibody. Then mutations (at the amino acid level) can be made in the isolated antibody, either incrementally, in combination, or both. For example, a nucleic acid library that includes sequences encoding some or all possible germline mutations is made. The mutated antibodies are then evaluated, e.g., to identify an antibody that has one or more additional germline residues relative to the isolated antibody and that is still useful (e.g., has a functional activity). In some embodiments, as many germline residues are introduced into an isolated antibody as possible.

In some embodiments, mutagenesis is used to substitute or insert one or more germline residues into a CDR region. For example, the germline CDR residue can be from a germline sequence that is similar (e.g., most similar) to the variable region being modified. After mutagenesis, activity (e.g., binding or other functional activity) of the antibody can be evaluated to determine if the germline residue or residues are tolerated. Similar mutagenesis can be performed in the framework regions.

Selecting a germline sequence can be performed in different ways. For example, a germline sequence can be selected if it meets a predetermined criterion for selectivity or similarity, e.g., at least a certain percentage identity, e.g., at least 75, 80, 85, 90, 91, 92, 93, 94, 95, 96, 97, 98, 99, or 99.5% identity, relative to the donor non-human antibody. The selection can be performed using at least 2, 3, 5, or 10 germline sequences. In the case of CDR1 and CDR2, identifying a similar germline sequence can include selecting one such sequence. In the case of CDR3, identifying a similar germline sequence can include selecting one such sequence, but may include using two germline sequences that separately contribute to the amino-terminal portion and the carboxy-terminal portion. In other implementations, more than one or two germline sequences are used, e.g., to form a consensus sequence.

Calculations of “sequence identity” between two sequences are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

In some embodiments, the antibody may be modified to have an altered glycosylation pattern (i.e., altered from the original or native glycosylation pattern). As used in this context, “altered” means having one or more carbohydrate moieties deleted, and/or having one or more glycosylation sites added to the original antibody. Addition of glycosylation sites to the presently disclosed antibodies may be accomplished by altering the amino acid sequence to contain glycosylation site consensus sequences; such techniques are well known in the art. Another means of increasing the number of carbohydrate moieties on the antibodies is by chemical or enzymatic coupling of glycosides to the amino acid residues of the antibody. These methods are described in, e.g., WO 87/05330, and Aplin and Wriston (1981) CRC Crit. Rev. Biochem., 22:259-306. Removal of any carbohydrate moieties present on the antibodies may be accomplished chemically or enzymatically as described in the art (Hakimuddin et al. (1987) Arch. Biochem. Biophys., 259:52; Edge et al. (1981) Anal. Biochem., 118:131; and Thotakura et al. (1987) Meth. Enzymol., 138:350). See, e.g., U.S. Pat. No. 5,869,046 for a modification that increases in vivo half-life by providing a salvage receptor binding epitope.

In some embodiments, an anti-ZNT8 antibody has one or more CDR sequences (e.g., a Chothia, an enhanced Chothia, or Kabat CDR) that differ from those described herein. In some embodiments, an anti-ZNT8 antibody has one or more CDR sequences include amino acid changes, such as substitutions of 1, 2, 3, or 4 amino acids if a CDR is 5-7 amino acids in length, or substitutions of 1, 2, 3, 4, or 5, of amino acids in the sequence of a CDR if a CDR is 8 amino acids or greater in length. The amino acid that is substituted can have similar charge, hydrophobicity, or stereochemical characteristics. In some embodiments, the amino acid substitution(s) is a conservative substitution. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine). In some embodiments, the amino acid substitution(s) is a non-conservative substitution. The antibody or antibody fragments thereof that contain the substituted CDRs can be screened to identify antibodies of interest.

Unlike in CDRs, more substantial changes in structure framework regions (FRs) can be made without adversely affecting the binding properties of an antibody. Changes to FRs include, but are not limited to, humanizing a nonhuman-derived framework or engineering certain framework residues that are important for antigen contact or for stabilizing the binding site, e.g., changing the class or subclass of the constant region, changing specific amino acid residues which might alter an effector function such as Fc receptor binding (Lund et al., J Immun., 147:26S7-62 (1991); Morgan et al., Immunology, 86:319-24 (199S)), or changing the species from which the constant region is derived.

V Humanized Antibodies A humanized antibody is a genetically engineered antibody in which the CDRs from a non-human “donor” antibody are grafted into human “acceptor” antibody sequences. See, e.g., Queen, U.S. Pat. Nos. 5,530,101 and 5,585,089; Winter, U.S. Pat. No. 5,225,539; Carter, U.S. Pat. No. 6,407,213; Adair, U.S. Pat. No. 5,859,205; and Foote, U.S. Pat. No. 6,881,557). The acceptor antibody sequences can be, for example, a mature human antibody sequence, a composite of such sequences, a consensus sequence of human antibody sequences, or a germline region sequence. In some embodiments, an acceptor sequence for the heavy chain is the germline V_{H} exon V_{H}1-2 (also referred to in the literature as HV1-2) (Shin et al, 1991, EMBO J. 10:3641-3645) and for the hinge region (JH), exon JH-6 (Mattila et al, 1995, Eur. J. Immunol. 25:2578-2582). For the light chain, an acceptor sequence can comprise exon VK2-30 (also referred to in the literature as KV2-30) and for the hinge region exon JK-4 (Hieter et al, 1982, J. Biol. Chem. 257:1516-1522). Thus, a humanized antibody is an antibody having some or all CDRs entirely or substantially from a donor antibody and variable region framework sequences and constant regions, if present, entirely or substantially from human antibody sequences. Similarly, a humanized heavy chain has at least one, two and usually all three CDRs entirely or substantially from a donor antibody heavy chain, and a heavy chain variable region framework sequence and heavy chain constant region, if present, substantially from human heavy chain variable region framework and constant region sequences. Similarly a humanized light chain has at least one, two and usually all three CDRs entirely or substantially from a donor antibody light chain, and a light chain variable region framework sequence and light chain constant region, if present, substantially from human light chain variable region framework and constant region sequences. Other than nanobodies and dAbs, a humanized antibody comprises a humanized heavy chain and a humanized light chain. A CDR in a humanized antibody is substantially from a corresponding CDR in a non-human antibody when at least 60%, 85%, 90%, 95% or 100% of corresponding residues (as defined by Kabat) are identical between the respective CDRs. The variable region framework sequences of an antibody chain or the constant region of an antibody chain are substantially from a human variable region framework sequence or human constant region respectively when at least 85%, 90%, 95% or 100% of corresponding residues defined by Kabat are identical. In some embodiments, the ZnT8 antibodies of the invention are humanized antibodies.

Although humanized antibodies often incorporate all six CDRs (preferably as defined by Kabat) from a mouse antibody, they can also be made with less than all CDRs (e.g., at least 3, 4, or 5) CDRs from a mouse antibody. See, e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; and Tamura et al, Journal of Immunology, 164:1432-1441, 2000.

The heavy and light chain variable regions of humanized antibodies can be linked to at least a portion of a human constant region. The choice of constant region depends, in part, whether antibody-dependent cell-mediated cytotoxicity, antibody dependent cellular phagocytosis and/or complement dependent cytotoxicity are desired. For example, human isotopes IgG1 and IgG3 have strong complement-dependent cytotoxicity, human isotype IgG2 weak complement-dependent cytotoxicity and human. IgG4 lacks complement-dependent cytotoxicity. Human IgG1 and IgG3 also induce stronger cell mediated effector functions than human IgG2 and IgG4. Light chain constant regions can be lambda or kappa. Antibodies can be expressed as tetramers containing two light and two heavy chains, as separate heavy chains, light chains, as Fab, Fab″, F(ab″)2, and Fv, or as single chain antibodies in which heavy and light chain variable domains are linked through a spacer.

Human constant regions show allotypic variation and isoallotypic variation between different individuals, that is, the constant regions can differ in different individuals at one or more polymorphic positions. Isoallotypes differ from allotypes in that sera recognizing an isoallotype binds to a non-polymorphic region of a one or more other isotypes. One or several amino acids at the amino or carboxy terminus of the light and/or heavy chain, such as the C-terminal lysine of the heavy chain, may be missing or derivatized in a proportion or all of the molecules. Substitutions can be made in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103:4005, 2006), or to prolong half-life in humans (see, e.g., Hinton et al., J. Biol. Chem. 279:6213, 2004).

Exemplary substitution include the amino acid substitution of the native amino acid to a cysteine residue is introduced at amino acid position 234, 235, 237, 239, 26T 298, 299, 326, 330, or 332, preferably an 5239C mutation in a human IgG1 isotype (US 20100158909). The presence of an additional cysteine residue allows interchain disulfide bond formation. Such interchain disulfide bond formation can cause steric hindrance, thereby reducing the affinity of the Fc region-FcyR binding interaction. The cysteine residue(s) introduced in or in proximity to the Fc region of an IgG constant region can also serve as sites for conjugation to therapeutic agents (i.e., coupling cytotoxic drugs using thiol specific reagents such as maleimide derivatives of drugs. The presence of a therapeutic agent causes steric hindrance, thereby further reducing the affinity of the Fc region-FcyR-binding interaction.

The in vivo half-life of an antibody can also impact on its effector functions. The half-life of an antibody can be increased or decreased to modify its therapeutic activities. FcRn is a receptor that is structurally similar to MHC Class I antigen that non-covalently associates with β2-microglobulin. FcRn regulates the catabolism of IgGs and their transcytosis across tissues (Ghetie and Ward, 2000, Annu. Rev. Immunol. 18:739-766; Ghetie and Ward, 2002, Immunol. Res. 25:97-113). The IgG-FcRn interaction takes place at pH 6.0 (pH of intracellular vesicles) but not at pH 7.4 (pH of blood); this interaction enables IgGs to be recycled back to the circulation (Ghetie and Ward, 2000, Ann. Rev. Immunol. 18:739-766; Ghetie and Ward, 2002, Immunol. Res. 25:97-113). The region on human IgG1 involved in FcRn binding has been mapped (Shields et al, 2001, J. Biol. Chem. 276:6591-604). Alanine substitutions at positions Pro238, Thr256, Thr307, Gln311, Asp312, Glu380, Glu382, or Asn434 of human IgG1 enhance FcRn binding (Shields et al, 2001, J. Biol. Chem. 276:6591-604). IgG1 molecules harboring these substitutions have longer serum half-lives. Consequently, these modified IgG1 molecules may be able to carry out their effector functions, and hence exert their therapeutic efficacies, over a longer period of time compared to unmodified IgG1. Other exemplary substitutions for increasing binding to FcRn include a Gin at position 250 and/or a Leu at position 428. EU numbering is used for all position in the constant region.

Reference to a human constant region includes a constant region with any natural allotype or any permutation of residues occupying polymorphic positions in natural allotypes. Also, up to 1, 2, 5, or 10 mutations may be present relative to a natural human constant region, such as those indicated above to reduce Fcgamma receptor binding or increase binding to FcRN.

### VI. Antibody-Drug Conjugates

Anti-ZnT8 antibodies can be conjugated to a therapeutic agent to form an antibody drug conjugate (ADC). In certain embodiments, the therapeutic agent can comprise cytotoxic agents, prodrug converting enzymes, radioactive isotopes or compounds, or toxins. For example, an anti-ZnT8 antibody can be conjugated to a cytotoxic agent such as a toxin (e.g., a cytostatic or cytocidal agent such as, e.g., abrin, ricin A, pseudomonas exotoxin, or diphtheria toxin).

An anti-ZnT8 antibody can be conjugated to a pro-drug converting enzyme. The pro-drug converting enzyme can be recombinantly fused to the antibody or chemically conjugated thereto using known methods. Exemplary pro-drug converting enzymes are carboxypeptidase G2, beta-glucuronidase, penicillin-V-amidase, penicillin-G-amidase, β-lactamase, β-glucosidase, nitroreductase and carboxypeptidase A.

Techniques for conjugating therapeutic agents to proteins, and in particular to antibodies, are well-known. See, e.g., Arnon et al, “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al, “Antibodies For Drug Delivery,” in Controlled Drug Delivery (Robinson et al. eds., Marcel Dekker, Inc., 2nd ed. 1987); Thorpe, “Antibody Carriers Of Cytotoxic Agents In Cancer Therapy: A Review,” in Monoclonal Antibodies '84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al, 1982, Immunol. Rev. 62:119-58. See also, e.g., PCT publication WO 89/12624.

The therapeutic agent can be conjugated in manner that reduces its activity unless it is cleaved off the antibody (e.g., by hydrolysis, by antibody degradation or by a cleaving agent). Such a therapeutic agent is attached to the antibody with a cleavable linker that is sensitive to cleavage in the intracellular environment of the ZnT8-expressing cancer cell but is not substantially sensitive to the extracellular environment, such that the conjugate is cleaved from the antibody when it is internalized by the ZnT8-expressing cell (e.g., in the endosomal or, for example by virtue of pH sensitivity or protease sensitivity, in the lysosomal environment or in the caveolear environment).

Typically the ADC comprises a linker region between the therapeutic agent and the anti-ZnT8 antibody. As noted supra, typically, the linker is cleavable under intracellular conditions, such that cleavage of the linker releases the therapeutic agent from the antibody in the intracellular environment (e.g., within a lysosome or endosome or caveolea). The linker can be, e.g., a peptidyl linker that is cleaved by an intracellular peptidase or protease enzyme, including a lysosomal or endosomal protease. Typically, the peptidyl linker is at least two amino acids long or at least three amino acids long. Most typical are peptidyl linkers that are cleavable by enzymes that are present in ZnT8-expressing cells. Other such linkers are described, e.g., in U.S. Pat. No. 6,214,345. In specific embodiments, the peptidyl linker cleavable by an intracellular protease comprises a Val-Cit linker or a Phe-Lys dipeptide (see, e.g., U.S. Pat. No. 6,214,345, which describes the synthesis of doxorubicin with the Val-Cit linker). One advantage of using intracellular proteolytic release of the therapeutic agent is that the agent is typically attenuated when conjugated and the serum stabilities of the conjugates are typically high.

The cleavable linker can be pH-sensitive, i.e., sensitive to hydrolysis at certain pH values. Typically, the pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. See, e.g., U.S. Pat. Nos. 5,122,368; 5,824,805; and 5,622,929; Dubowchik and Walker, 1999, Pharm. Therapeutics 83:67-123; Neville et al, 1989, Biol. Chem. 264: 14653-14661. Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the therapeutic agent via an acylhydrazone bond (see, e.g., U.S. Pat. No. 5,622,929)).

Other linkers are cleavable under reducing conditions (e.g., a disulfide linker). Disulfide linkers include those that can be formed using SATA (N-succinimidyl-S-acetylthioacetate), SPDP (N-succinimidyl-3-(2-pyridyldithio)propionate), SPDB (N-succininmidyl-3-(2-pyridyldithio)butyrate) and SMPT (N-succinimidyl-oxycarbonyl-alpha-methyl-alpha-(2-pyridyl-dithio)toluene), SPDB and SMPT. See, e.g., Thorpe et al, 1987, Cancer Res. 47:5924-5931; Wawrzynczak et al, In Immunoconjugates: Antibody Conjugates in Radioimagery and Therapy of Cancer (C. W. Vogel ed., Oxford U. Press, 1987). See also U.S. Pat. No. 4,880,935.

The linker can also be a malonate linker (Johnson et al, 1995, Anticancer Res. 15:1387-93), a maleimidobenzoyl linker (Lau et al, 1995, Bioorg-Med-Chem. 3(10):1299-1304), or a 3′-N-amide analog (Lau et al, 1995. Bioorg-Med-Chem. 3(10):1305-12). The linker can also be a malonate linker (Johnson et al, 1995, Anticancer Res. 15:1387-93), a maleimidobenzoyl linker (Lau et al, 1995, Bioorg-Med-Chem 0.3(10):1299-1304), or a 3′-N-amide analog (Lau et al, 1995, Bioorg-Med-Chem. 3(10):1305-12).

The linker also can be a non-cleavable linker, such as a maleimido-alkylene- or maleimide-aryl linker that is directly attached to the therapeutic agent (e.g., a drug). An active drug-linker is released by degradation of the antibody.

Typically, the linker is not substantially sensitive to the extracellular environment meaning that no more than about 20%, typically no more than about 15%, more typically no more than about 10%, and even more typically no more than about 5%, no more than about 3%, or no more than about 1% of the linkers in a sample of the ADC is cleaved when the ADC present in an extracellular environment (e.g., in plasma).

Whether a linker is not substantially sensitive to the extracellular environment can be determined, for example, by incubating independently with plasma both (a) the ADC (the “ADC sample”) and (b) an equal molar amount of unconjugated antibody or therapeutic agent (the “control sample”) for a predetermined time period (e.g., 2, 4, 8, 16, or 24 hours) and then comparing the amount of unconjugated antibody or therapeutic agent present in the ADC sample with that present in control sample, as measured, for example, by high performance liquid chromatography.

The linker can also promote cellular internalization. The linker can promote cellular internalization when conjugated to the therapeutic agent (i.e. in the milieu of the linker-therapeutic agent moiety of the ADC or ADC derivative as described herein). Alternatively, the linker can promote cellular internalization when conjugated to both the therapeutic agent and the anti-ZnT8 antibody (i.e., in the milieu of the ADC as described herein).

The anti-ZnT8 antibody can be conjugated to the linker via a heteroatom of the antibody. These heteroatoms can be present on the antibody in its natural state or can be introduced into the antibody. In some aspects, the anti-ZnT8 antibody will be conjugated to the linker via a nitrogen atom of a lysine residue. In other aspects, the anti-ZnT8 antibody will be conjugated to the linker via a sulfur atom of a cysteine residue. The cysteine residue can be naturally-occurring or one that is engineered into the antibody. Methods of conjugating linkers and drug-linkers to antibodies via lysine and cysteine residues are known in the art.

### VII. Imaging

In another aspect, the antibody is conjugated to a labeling agent. By “labeling agent” (or “detectable label”) is meant the agent detectably labels the antibody, such that the antibody may be detected in an application of interest (e.g., in vitro and/or in vivo research and/or clinical applications). Detectable labels of interest include radioisotopes, enzymes that generate a detectable product (e.g., horseradish peroxidase, alkaline phosphatase, etc.), fluorescent proteins, paramagnetic atoms, and the like. In certain aspects, the antibody is conjugated to a specific binding partner of detectable label (e.g., conjugated to biotin such that detection may occur via a detectable label that includes avidin/streptavidin).

In certain embodiments, the agent is a labeling agent that finds use in in vivo imaging such as, but not limited to, near-infrared (NIR) optical imaging, single-photon emission computed tomography (SPECT)/CT imaging, positron emission tomography (PET), nuclear magnetic resonance (NMR) spectroscopy, and the like. Labeling agents that find use in such applications include, but are not limited to, fluorescent labels, radioisotopes, and the like. In particular embodiments, the labeling agent is a multi-modal in vivo imaging agent that permits in vivo imaging using two or more imaging approaches. See Thorp-Greenwood and Coogan (2011) Dalton Trans. 40:6129-6143. In other embodiment, the labeling agent is an in vivo imaging agent that finds use in near-infrared (NIR) imaging applications, which agent is selected from a Kodak X-SIGHT dye, Pz 247, DyLight 750 and 800 Fluors, Cy 5.5 and 7 Fluors, Alexa Fluor 680 and 750 Dyes, IRDye 680 and 800CW Fluors. In some embodiments, the labeling agent is an in vivo imaging agent that finds use in SPECT imaging applications, which agent can include, but is not limited to, ^{99m}Tc, In-111, 123-In, ²⁰¹Tl, and ¹³³Xe. In specific embodiments, the labeling agent is an in vivo imaging agent that finds use in positron emission tomography (PET) imaging applications, which agent can include, but is not limited to, ^{U}C, ¹³N, ¹⁵O, ¹⁸F, ⁶⁴Cu, ⁶²Cu, ¹²⁴I, ⁷⁶Br, ⁸²Rb and ⁶⁸Ga.

### VIII. Methods of Producing Anti-ZNT8 Antibodies

The anti-ZNT8 antibodies (or antigen binding domain(s) of an antibody or functional fragment thereof) of this disclosure may be produced in bacterial or eukaryotic cells. To produce the polypeptide of interest, a polynucleotide encoding the polypeptide is constructed, introduced into an expression vector, and then expressed in suitable host cells. Standard molecular biology techniques are used to prepare the recombinant expression vector, transfect the host cells, select for transformants, culture the host cells and recover the antibody.

If the antibody is to be expressed in bacterial cells (e.g., E. coli), the expression vector should have characteristics that permit amplification of the vector in the bacterial cells. Additionally, when E. coli such as JM109, DH5a, HBlOl, or XL I-Blue is used as a host, the vector must have a promoter, for example, a lacZ promoter (Ward et al., 341:544-546 (1989), araB promoter (Better et al., Science, 240: 1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, 293, 293T, and NIH3T3 cells, the expression vector includes a promoter necessary for expression in these cells, for example, an SV40 promoter (Mulligan et al., Nature, 277:108 (1979)), MMLV-LTR promoter, EF1a promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In some embodiments, the antibodies are produced in mammalian cells. Exemplary mammalian host cells for expressing a polypeptide include Chinese Hamster Ovary (CHO cells) (including dhfr-CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601 621), human embryonic kidney 293 cells (e.g., 293, 293E, 293T), COS cells, NIH3T3 cells, lymphocytic cell lines, e.g., NSO myeloma cells and SP2 cells, and a cell from a transgenic animal, e.g., a transgenic mammal. For example, the cell is a mammary epithelial cell.

The antibodies of the present disclosure can be isolated from inside or outside (such as medium) of the host cell and purified as substantially pure and homogenous antibodies. Methods for isolation and purification commonly used for polypeptides may be used for the isolation and purification of antibodies described herein, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

The present disclosure also provides a nucleic acid molecule or a set of nucleic acid molecules encoding an anti-ZNT8 antibody or antigen binding molecule thereof disclosed herein. In some embodiments, the invention includes a nucleic acid molecule encoding a polypeptide chain, which comprises a light chain of an anti-ZNT8 antibody or antigen-binding molecule thereof as described herein. In some embodiments, the invention includes a nucleic acid molecule encoding a polypeptide chain, which comprises a heavy chain of an anti-ZNT8 antibody or antigen-binding molecule thereof as described herein.

Also provided are a vector or a set of vectors comprising such nucleic acid molecule or the set of the nucleic acid molecules or a complement thereof, as well as a host cell comprising the vector.

The instant disclosure also provides a method for producing a ZNT8 or antigen-binding molecule thereof or chimeric molecule disclosed herein, such method comprising culturing the host cell disclosed herein and recovering the antibody, antigen-binding molecule thereof, or the chimeric molecule from the culture medium.

A variety of methods are available for recombinantly producing a ZNT8 antibody or antigen-binding molecule thereof disclosed herein, or a chimeric molecule disclosed herein. It will be understood that because of the degeneracy of the code, a variety of nucleic acid sequences will encode the amino acid sequence of the polypeptide. The desired polynucleotide can be produced by de novo solid-phase DNA synthesis or by PCR mutagenesis of an earlier prepared polynucleotide.

For recombinant production, a polynucleotide sequence encoding a polypeptide (e.g., a ZNT8 antibody or antigen-binding molecule thereof disclosed herein, or any of the chimeric molecules disclosed herein) is inserted into an appropriate expression vehicle, i.e., a vector which contains the necessary elements for the transcription and translation of the inserted coding sequence, or in the case of an RNA viral vector, the necessary elements for replication and translation.

The nucleic acid encoding the polypeptide (e.g., a ZNT8 antibody or antigen-binding molecule thereof disclosed herein, or any of the chimeric molecules disclosed herein) is inserted into the vector in proper reading frame. The expression vector is then transfected into a suitable target cell which will express the polypeptide. Transfection techniques known in the art include, but are not limited to, calcium phosphate precipitation (Wigler et al. 1978, Cell 14:725) and electroporation (Neumann et al. 1982, EMBO J. 1:841). A variety of host-expression vector systems can be utilized to express the polypeptides described herein (e.g., a ZNT8 antibody or antigen-binding molecule thereof disclosed herein, or any of the chimeric molecules disclosed herein) in eukaryotic cells. In some embodiments, the eukaryotic cell is an animal cell, including mammalian cells (e.g., 293 cells, PerC6, CHO, BHK, Cos, HeLa cells). When the polypeptide is expressed in a eukaryotic cell, the DNA encoding the polypeptide (e.g., a ZNT8 antibody or antigen-binding molecule thereof disclosed herein, or any of the chimeric molecules disclosed herein) can also code for a signal sequence that will permit the polypeptide to be secreted. One skilled in the art will understand that while the polypeptide is translated, the signal sequence is cleaved by the cell to form the mature chimeric molecule. Various signal sequences are known in the art and familiar to the skilled practitioner. Alternatively, where a signal sequence is not included, the polypeptide (e.g., a ZNT8 antibody or antigen-binding molecule thereof disclosed herein, or any of the chimeric molecules disclosed herein) can be recovered by lysing the cells.

### IX. Pharmaceutical Compositions

The present disclosure also provides pharmaceutical compositions comprising one or more of: (i) a ZNT8 antibody or antigen-binding molecule thereof disclosed herein; (ii) a nucleic acid molecule or the set of nucleic acid molecules encoding a ZNT8 antibody or antigen-binding molecule as disclosed herein; or (iii) a vector or set of vectors disclosed herein, and a pharmaceutically acceptable carrier.

Anti-ZNT8 antibodies or fragments thereof described herein can be formulated as a pharmaceutical composition for administration to a subject, e.g., to treat a disorder described herein. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or abase addition salt (see e.g., Berge, S. M., et al. (1977) J. Pharm. Sci. 66:1-19).

Pharmaceutical formulation is a well-established art, and is further described, e.g., in Gennaro (ed.), Remington: The Science and Practice of Pharmacy, 20th ed., Lippincott, Williams & Wilkins (2000) (ISBN: 0683306472); Ansel et al., Pharmaceutical Dosage Forms and Drug Delivery Systems, 7th Ed., Lippincott Williams & Wilkins Publishers (1999) (ISBN: 0683305727); and Kibbe (ed.), Handbook of Pharmaceutical Excipients American Pharmaceutical Association, 3rd ed. (2000) (ISBN: 091733096X).

The pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application. Typically compositions for the agents described herein are in the form of injectable or infusible solutions.

In some embodiments, an antibody described herein is formulated with excipient materials, such as sodium citrate, sodium dibasic phosphate heptahydrate, sodium monobasic phosphate, Tween®-80, and a stabilizer. It can be provided, for example, in a buffered solution at a suitable concentration and can be stored at 2-8° C. In some embodiments, the pH of the composition is between about 5.5 and 7.5 (e.g., 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, and 7.5).

The pharmaceutical compositions can also include agents that reduce aggregation of the antibody when formulated. Examples of aggregation reducing agents include one or more amino acids selected from the group consisting of methionine, arginine, lysine, aspartic acid, glycine, and glutamic acid. These amino acids may be added to the formulation to a concentration of about 0.5 mM to about 145 mM (e.g., 0.5 mM, 1 mM, 2 mM, 5 mM, 10 mM, 25 mM, 50 mM, 100 mM). The pharmaceutical compositions can also include a sugar (e.g., sucrose, trehalose, mannitol, sorbitol, or xylitol) and/or a tonicity modifier (e.g., sodium chloride, mannitol, or sorbitol) and/or a surfactant (e.g., polysorbate-20 or polysorbate-80).

The composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization.

Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, the antibodies may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York (1978).

In some embodiments, the pharmaceutical formulation comprises an antibody at a concentration of about 0.005 mg/mL to 500 mg/mL (e.g., 0.005 mg/ml, 0.01 mg/ml, 0.05 mg/ml, 0.1 mg/ml, 0.5 mg/mL, 1 mg/mL, 5 mg/mL, 10 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, 125 mg/mL, 150 mg/mL, 175 mg/mL, 200 mg/mL, 250 mg/mL, 300 mg/mL, 350 mg/mL, 400 mg/mL, 450 mg/mL, 500 mg/mL), formulated with a pharmaceutically acceptable carrier. In some embodiments, the antibody is formulated in sterile distilled water or phosphate buffered saline. The pH of the pharmaceutical formulation may be between 5.5 and 7.5 (e.g., 5.5, 5.6, 5.7, 5.8, 5.9, 6.0, 6.1, 6.2 6.3, 6.4 6.5, 6.6 6.7, 6.8, 6.9 7.0, 7.1, 7.3, 7.4, 7.5).

A pharmaceutical composition may include a “therapeutically effective amount” of an agent described herein. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition are outweighed by the therapeutically beneficial effects.

The antibodies or antigen-binding fragment thereof, or nucleic acids encoding same of the disclosure can be administered to a subject, e.g., a subject in need thereof, for example, a human or animal subject, by a variety of methods. For many applications, the route of administration is one of intravenous injection or parenteral, infusion (IV), subcutaneous injection (SC), intraperitoneally (IP), or intramuscular injection, intratumor (IT). Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and intrasternal injection.

In some embodiments, the route of administration of the antibodies of the invention is parenteral. The term parenteral as used herein includes intravenous, intraarterial, intraperitoneal, intramuscular, subcutaneous, rectal or vaginal administration. The intravenous form of parenteral administration is preferred. While all these forms of administration are clearly contemplated as being within the scope of the invention, a form for administration would be a solution for injection, in particular for intravenous or intraarterial injection or drip. Usually, a suitable pharmaceutical composition for injection can comprise a buffer (e.g., acetate, phosphate or citrate buffer), a surfactant (e.g., polysorbate), optionally a stabilizer agent (e.g., human albumin), etc. However, in other methods compatible with the teachings herein, the polypeptides can be delivered directly to the site of the adverse cellular population thereby increasing the exposure of the diseased tissue to the therapeutic agent.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

Pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a polypeptide by itself or in combination with other active agents) in the required amount in an appropriate solvent with one or a combination of ingredients enumerated herein, as required, followed by filtered sterilization.

Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations can be packaged and sold in the form of a kit. Such articles of manufacture will preferably have labels or package inserts indicating that the associated compositions are useful for treating a subject suffering from, or predisposed to clotting disorders.

Effective doses of the compositions of the present disclosure, for the treatment of conditions vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages can be titrated using routine methods known to those of skill in the art to optimize safety and efficacy.

The route and/or mode of administration of the anti-ZNT8 antibody or fragment thereof can also be tailored for the individual case, e.g., by monitoring the subject.

The antibody or fragment thereof can be administered as a fixed dose, or in a mg/kg dose. The dose can also be chosen to reduce or avoid production of antibodies against the anti-ZNT8 antibody or fragment thereof. Dosage regimens are adjusted to provide the desired response, e.g., a therapeutic response or a combinatorial therapeutic effect. Generally, doses of the antibody or fragment thereof (and optionally a second agent) can be used in order to provide a subject with the agent in bioavailable quantities. For example, doses in the range of 0.1-100 mg/kg, 0.5-100 mg/kg, 1 mg/kg-100 mg/kg, 0.5-20 mg/kg, 0.1-10 mg/kg, or 1-10 mg/kg can be administered. Other doses can also be used. In certain embodiments, a subject in need of treatment with an antibody or fragment thereof is administered the antibody or fragment thereof at a dose of between about 1 mg/kg to about 30 mg/kg. In some embodiments, a subject in need of treatment with anti-ZNT8 antibody or fragment thereof is administered the antibody or fragment thereof at a dose of 1 mg/kg, 2 mg/kg, 4 mg/kg, 5 mg/kg, 7 mg/kg 10 mg/kg, 12 mg/kg, 15 mg/kg, 20 mg/kg, 25 mg/kg, 28 mg/kg, 30 mg/kg, 35 mg/kg, 40 mg/kg, or 50 mg/kg. In a specific embodiment, the antibody or fragment thereof is administered subcutaneously at a dose of 1 mg/kg to 3 mg/kg. In another embodiment, the antibody or fragment thereof is administered intravenously at a dose of between 4 mg/kg and 30 mg/kg.

A composition may comprise about 1 mg/mL to 100 mg/ml or about 10 mg/mL to 100 mg/ml or about 50 to 250 mg/mL or about 100 to 150 mg/ml or about 100 to 250 mg/ml of the antibody or fragment thereof.

Dosage unit form or “fixed dose” as used herein refers to physically discrete units suited as unitary dosages for the subjects to be treated; each unit contains a predetermined quantity of antibody or fragment thereof calculated to produce the desired therapeutic effect in association with the required pharmaceutical carrier and optionally in association with the other agent. Single or multiple dosages may be given. Alternatively, or in addition, the antibody or fragment thereof may be administered via continuous infusion.

An antibody or fragment thereof dose can be administered, e.g., at a periodic interval over a period of time (a course of treatment) sufficient to encompass at least 2 doses, 3 doses, 5 doses, 10 doses, or more, e.g., once or twice daily, or about one to four times per week, or preferably weekly, biweekly (every two weeks), every three weeks, monthly, e.g., for between about 1 to 12 weeks, preferably between 2 to 8 weeks, more preferably between about 3 to 7 weeks, and even more preferably for about 4, 5, or 6 weeks. Factors that may influence the dosage and timing required to effectively treat a subject, include, e.g., the stage or severity of the disease or disorder, formulation, route of delivery, previous treatments, the general health and/or age of the subject, and other diseases present. Moreover, treatment of a subject with a therapeutically effective amount of a compound can include a single treatment or, preferably, can include a series of treatments.

If a subject is at risk for developing a disorder described herein, the antibody or fragment thereof can be administered before the full onset of the disorder, e.g., as a preventative measure. The duration of such preventative treatment can be a single dosage of the antibody or fragment thereof or the treatment may continue (e.g., multiple dosages). For example, a subject at risk for the disorder or who has a predisposition for the disorder may be treated with the antibody or fragment thereof for days, weeks, months, or even years so as to prevent the disorder from occurring or fulminating.

In certain embodiments, the antibody or fragment thereof is administered subcutaneously at a concentration of about 1 mg/mL to about 500 mg/mL (e.g., 1 mg/mL, 2 mg/mL, 3 mg/mL, 4 mg/mL, 5 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, 125 mg/mL, 150 mg/mL, 175 mg/mL, 200 mg/mL, 225 mg/mL, 250 mg/mL, 275 mg/mL, 300 mg/mL, 325 mg/mL, 350 mg/mL, 400 mg/mL, 450 mg/mL). In some embodiments, the anti-ZNT8 antibody or fragment thereof is administered subcutaneously at a concentration of 50 mg/mL. In another embodiment, the antibody or fragment thereof is administered intravenously at a concentration of about 1 mg/mL to about 500 mg/mL. In some embodiments, the antibody or fragment thereof is administered intravenously at a concentration of 50 mg/mL.

Doses intermediate in the above ranges are also intended to be within the scope of the invention. Subjects can be administered such doses daily, on alternative days, weekly or according to any other schedule determined by empirical analysis. An exemplary treatment entails administration in multiple dosages over a prolonged period, for example, of at least six months. In some methods, two or more polypeptides can be administered simultaneously, in which case the dosage of each polypeptide administered falls within the ranges indicated.

Polypeptides of the invention can be administered on multiple occasions. Intervals between single dosages can be daily, weekly, monthly or yearly. Intervals can also be irregular as indicated by measuring blood levels of modified polypeptide or antigen in the patient. Alternatively, polypeptides can be administered as a sustained release formulation, in which case less frequent administration is required. Dosage and frequency vary depending on the half-life of the polypeptide in the patient.

The dosage and frequency of administration can vary depending on whether the treatment is prophylactic or therapeutic. In prophylactic applications, compositions containing the polypeptides of the invention or a cocktail thereof are administered to a patient not already in the disease state to enhance the patient's resistance or minimize effects of disease. Such an amount is defined to be a “prophylactic effective dose.” A relatively low dosage is administered at relatively infrequent intervals over a long period of time. Some patients continue to receive treatment for the rest of their lives.

### X. Devices and Kits for Therapy

An anti-ZNT8 antibody or fragment thereof can be provided in a kit. In some embodiments, the kit includes (a) a container that contains a composition that includes an anti-ZNT8 antibody or fragment thereof as described herein, and optionally (b) informational material. The informational material can be descriptive, instructional, marketing or other material that relates to the methods described herein and/or the use of the agents for therapeutic benefit.

In certain embodiments, the kit also includes a second agent for treating a disorder described herein, i.e., a disease or condition mediated by or associated with ZnT8 (e.g., Type 1 or Type 2 diabetes). For example, the kit includes a first container that contains a composition that includes the anti-ZNT8 antibody or fragment thereof, and a second container that includes the second agent.

In some embodiments, the kit also includes a second agent such as an imaging agent. For example, the kit includes a first container that contains a composition that includes the anti-ZNT8 antibody or fragment thereof, and a second container that includes the second agent.

The informational material of the kits is not limited in its form. In some embodiments, the informational material can include information about production of the compound, molecular weight of the compound, concentration, date of expiration, batch or production site information, and so forth. In some embodiments, the informational material relates to methods of administering the anti-ZNT8 antibody or fragment thereof, e.g., in a suitable dose, dosage form, or mode of administration (e.g., a dose, dosage form, or mode of administration described herein), to treat a subject who has had or who is at risk for a disease as described herein. The information can be provided in a variety of formats, include printed text, computer readable material, video recording, or audio recording, or information that provides a link or address to substantive material, e.g., on the internet.

In addition to the anti-ZNT8 antibody or fragment thereof, the composition in the kit can include other ingredients, such as a solvent or buffer, a stabilizer, or a preservative. The anti-ZNT8 antibody or fragment thereof can be provided in any form, e.g., liquid, dried or lyophilized form, preferably substantially pure and/or sterile. When the agents are provided in a liquid solution, the liquid solution preferably is an aqueous solution. In certain embodiments, the anti-ZNT8 antibody or fragment thereof in the liquid solution is at a concentration of about 25 mg/mL to about 250 mg/mL (e.g., 40 mg/mL, 50 mg/mL, 60 mg/mL, 75 mg/mL, 85 mg/mL, 100 mg/mL, 125 mg/mL, 150 mg/mL, and 200 mg/mL). When the anti-ZNT8 antibody or fragment thereof is provided as a lyophilized product, the anti-ZNT8 antibody or fragment thereof is at about 75 mg/vial to about 200 mg/vial (e.g., 100 mg/vial, 108.5 mg/vial, 125 mg/vial, 150 mg/vial). The lyophilized powder is generally reconstituted by the addition of a suitable solvent. The solvent, e.g., sterile water or buffer (e.g., PBS), can optionally be provided in the kit.

The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In some embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., a unit that includes both the anti-ZNT8 antibody or fragment thereof and the second agent, e.g., in a desired ratio. For example, the kit includes a plurality of syringes, ampules, foil packets, blister packs, or medical devices, e.g., each containing a single combination unit dose. The containers of the kits can be airtight, waterproof (e.g., impermeable to changes in moisture or evaporation), and/or light-tight.

The kit optionally includes a device suitable for administration of the composition, e.g., a syringe or other suitable delivery device. The device can be provided pre-loaded with one or both of the agents or can be empty, but suitable for loading.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1: Generation and Characterization of mAb43

### Materials and Methods

Animals. NOD, C57BL/6 and MIP-GFP mice were purchased from Jackson Laboratory and ZnT8-KO mice from Taconic. Mice were maintained in group housing in sterile containers within a pathogen-free barrier facility housed with a 12 hr light/12 hr dark cycle and free access to water and standard rodent chow. All animal procedures were approved by the Institutional Animal Care and Use Committees of Johns Hopkins University School of Medicine, and Barbara Davis Center for Diabetes, University of Colorado.

Production of human ZnT8 antigen and proteoliposome reconstitution. Human ZnT8 isoform-2 cDNA (NM_001172814.1) was subcloned into a mammalian pCMV6-based expression vector with a C-terminal His-tag (16). The expression plasmid was introduced into FreeStyle 293-F cells and transiently expressed in suspension culture of a serum-free medium per manufacturer's instructions. Human CTD-His was constructed by a N-terminal deletion to remove the entire TMD sequence from the ZnT8-His construct, and transiently expressed in 293-F cells as above. Cells expressing either ZnT8-His or CTD-His were harvested 18 hours post-transfection, and then homogenized using a microfluidizer. The cellular membrane was separated from the cytosolic fraction by ultracentrifugation. The membrane-bound ZnT8-His was detergent extracted and purified as described previously (16). The purified ZnT8-His was reconstituted at a ZnT8/lipid ratio of 1/20 (wt/wt) into proteoliposomes composed of DOPC, DOPE and DOPG at a 2:1:1 ratio. Lipid-A adjuvant was added to the reconstitution lipid mixture to a concentration of 10% of the total lipid content. The reconstituted ZnT8-His in proteoliposomes remained functionally active and could be re-solubilized by detergent to form a monodispersed species on sizing HPLC (26). Liposomes were prepared in parallel to proteoliposomes without adding ZnT8-His to the lipid reconstitution mixture.

Mouse immunization and mAb43 generation. Four pairs of seven-week-old male/female homozygous ZnT8-KO mice were used for proteoliposome immunization and a single pair of male/female littermates for liposome immunization. Five NOD females at 10 weeks of age were used for proteoliposome immunization and three NOD female littermates for liposome immunization. Each mouse received weekly intraperitoneal injections of 50-60 μg purified ZnT8 in proteoliposome emulsion or in an equal volume of liposome emulsion (100 μl). Submental bleeds were collected three weeks post-injection and used for serum antibody titering by comparative ZnT8 and CTD ELISAs. All mice were euthanized five weeks post injection. Draining lymph nodes and spleens were collected to generate hybridoma fusions by electrofusion. The fused cells were HAT selected and cloned in a semi-solid ClonaCell™-HY Medium D, expanded in Medium E in 96-well plates for mAb screening by comparative ELISAs (see below). A dozen positive clones were expanded in AOF medium for large-scale mAb production. Cell-culture grade mAbs were produced by size-exclusion HPLC purification in PBS, and used for live cell screening based on ZnT8 binding on the surface of INS-1E cells stably expressing human ZnT8-GFP (11). The variable regions of the mAb43 transcript in the hybridoma cell were sequenced, and subcloned into a mammalian bicistronic IRES expression vector carrying human signal peptide, kappa and gamma constant regions (TakaraBio, pIRES Vector; Addgene, pVITRO1-dV-IgG1/κ; pVITRO1-Trastuzumab-IgG2/x; pVITRO1-Trastuzumab-IgG3/x; pVITRO1-Trastuzumab-IgG4/x). The recombinant mAb43 constructs of various IgG isotypes were transiently expressed in 293-F cells, then purified and validated for ZnT8 binding based on the formation of stable mAb43-ZnT8-GFP complexes on fluorescence size-exclusion HPLC.

Comparative ELISAs. For proteoliposome-based ELISA, 4 μg proteoliposomes (containing 5% human ZnT8-His by weight) diluted in 100 μl PBS were added to each well of a high-binding 96-well plate, and incubated overnight at 4° C. The passively immobilized proteoliposomes were blocked with 5% BSA, and then tested with hybridoma culture supernatants. For solution-based ELISA, 293-F cells expressing human ZnT8-His or CTD-His were mechanically lysed using a microfluidizer, and cleared of cell debris by ultracentrifugation. Then, 0.2 μg human ZnT8-His from detergent-solubilized cell membrane or 0.1 μg CTD-His from cell lysate in 100 μl PBS were immobilized to each well of a nickel-coated 96-well plate via the C-terminal His-tag. The immobilized protein was blocked by 5% BSA, and then tested with mouse sera in 3-fold serial dilutions or hybridoma culture supernatants. Bound serum antibodies were detected by an HRP-conjugated goat anti-mouse IgG secondary antibody (1:3000) on a Flexstation-3 microplate reader.

Immunofluorescence labeling and imaging analysis. EndoC-βH1 cells were seeded onto a glass bottom microwell dish that was pre-coated with β-coat and grown in OPTI cell culture medium at 37° C. in a 5% CO2 humidified atmosphere for two days. For cell surface IF-labeling, live cells were washed with a high glucose (20 mM) Krebs buffer, chilled at 8° C. for 30 min, and then exposed to mAb43 (1:100), mAb20 (1:100), anti-CD71 (1:50) or anti-Na+/K+ ATPase (1:50) antibody. After 1 hr incubation at 8° C., unbound antibodies were removed by 2× wash using high glucose Krebs buffer. Next, cells were exposed to a fluorescent anti-IgG secondary antibody (1:400) for 0.5 hr, washed free of unbound secondary antibody, and then DAPI/DCV was added to the medium for fluorescence imaging on a Zeiss LSM 700 inverted confocal microscope with a 63× oil objective. For intracellular IF labeling, live cells were washed with a high glucose (20 mM) Krebs buffer, fixed using a flowcytometry fixation buffer for 20 min at RT, washed again using PBS, permeabilized with flowcytometry permeabilization buffer for 20 min at RT, blocked with PBS plus 5% BSA for 30 min, and then exposed to mAb43 (1:1000), mAb20 (1:1000), anti-CD71 (1:200) or anti-Na+/K+ ATPase (1:200) antibody for 2 hr at room temperature. Secondary antibody immunolabeling, DAPI counterstain and immunofluorescence imaging were performed using the same procedure as above. For experiments with wild-type INS-1E cells or ZnT8-KO INS-1E cells (13), cells were grown in RMPI 1640 medium supplied with 10% (v/v) fetal bovine serum (FBS), 100 units/ml penicillin, 100 μg/ml streptomycin, 10 mM HEPES, 2 mM glutamine, 1 mM sodium pyruvate and 50 μM β-ME. Immunofluorescence labeling and imaging followed the same procedure as above. For antibody internalization in EndoC-βH1 cells, mAb43 (1: 100) and NTPDase3 (1:100) were first co-incubated with respective Alexa fluor-647 (1:200) and Alexa fluor-488 secondary antibodies (1:200) to form fluorescent antibody complexes, and then added to live EndoC-βH1 cells at 37° C. for 1 hr before IF imaging.

Immunohistochemistry. Excised mouse pancreas was fixed in 4% PFA at 4° C. for 4 hr, processed and then embedded in paraffin. Tissue sections (4 μm) were dewaxed and rehydrated, blocked for one hours, then incubated with chimeric mAb43 or chimeric mAb20 at 1:50 in a universal antibody dilution buffer at 4° C. for 16 hr, followed by a secondary biotinylated anti-human IgG antibody (1:400) for 30 minutes at 37° C., and then avidin biotinylated-peroxidase complex for 30 min at 37° C. Next, diaminobenzidine substrate was applied to develop optimal staining intensity. The colorimetric reaction was terminated by washing with dH2O. Next, pancreas sections were counterstained with eosin, dehydrated and mounted with xylene-compatible mounting medium for imaging.

Fluorescence size-exclusion HPLC analysis. Approximately 3×106 stably transfected INS-1E cells expressing ZnT8-GFP or ZnT8FLAG-GFP were solubilized using 200 μl assay buffer (20 mM HEPES, 100 mM NaCl, pH 7.0) plus 0.5% DDM. The detergent crude extract containing ZnT8-GFP or ZnT8FLAG-GFP was injected into a size-exclusion TSK HPLC column and monitored for GFP-fluorescence using a fluorescence detector (488/510 nm). ZnT8-GFP was collected as a monodispersed peak fraction. Next, the HPLC isolated ZnT8-GFP or ZnT8FLAG-GFP was incubated with mAb43, mAb20 or anti-FLAG antibody for 1 hour on ice, and then re-injected into the HPLC column. The ZnT8-antibody complex was collected for immunoblotting analysis to validate the presence of both ZnT8 and antibody in the binding complex.

Purification of ZnT8-Fab43 and EM single partial analysis. mAb43 was produced by hybridoma cells grown in a serum-free AOF medium for 3 weeks, captured by protein A/G beads, eluted by an IgG elution buffer, and concentrated to −20 mg/ml for Fab production using a Piercers Fab preparation kit following manufacturer's protocol. The purified Fab43 was mixed with purified ZnT8 in reconstituted proteoliposomes at 5:1 molar ratio plus 1% DDM to solubilize Fab43-ZnT8 in a lipid-rich detergent solution. The Fab43-ZnT8 complex was polished through a TSK size-exclusion HPLC column equilibrized with 0.05% DDM in 20 mM HEPES and 100 mM NaCl, pH=7.0. After three runs of HPLC delipidation, the ZnT8-Fab complex was collected in a monodispersed elution peak. The purified protein sample was diluted to 20 μg/ml, and aliquots of 3 μl diluted sample were applied on glow-discharged EM grids covered with a continuous thin carbon film and stained by 2% uranyl formate aqueous solution for 0.5 min. Grids were loaded onto a Tecnai Spirit electron microscope operated at a high tension of 120 kV. Electron micrographs were recorded in low-dose mode (10 e-/Å) using a Gatan Orius CCD camera with an under-focus value ranging from 1 to 2.5 μm and at a magnification of 30,000, which corresponded to 2.3 Å/pixel at the specimen level. A total of 92 micrographs was collected, and the contrast transfer function parameters of each image were determined by CTFFIND4.1.10. 12,778 particles were picked from the micrographs. After 2D classification in RELION3.0 and 3D classification in cryoSPARC3.1, 9,216 particle images were retained for 3D reconstruction. 3D refinement was performed using cryoSPARC3.1, yielding a 3D EM map at an estimated resolution of 1.5 nm. The present inventors used the Fab structure (PDB 1M71), cryo-EM structure of human ZnT8 (PDB 6XPD) and rigid-body docking to fit component structures into the EM map of the ZnT8-Fab43 complex.

Tissue dispersion and pancreatic cell labeling. Excised pancreata from C57BL/6 mice were cut into small pieces, minced, and washed with HBSS on a 70-μm strainer to remove hematopoietic cells. The washed tissue pellets were resuspended in accutase and incubated at 37° C. for 30 min. DCV was added to stain DNA of live cells. The dispersed cells were filtrated through the strainer by a gentle spin at 1200 rpm for 2 min. The remaining tissue pellets underwent additional cycles of accutase digestion and cell filtration to achieve a complete cell dispersion. The dispersed cells were pooled and washed with cold cell culture medium with DNase and trypsin/chymotrypsin inhibitors. At this point, cell viability, measured by trypan blue exclusion, was typically over 80%. Dispersed cells were adjusted to a cell density of 106/100 μl in flow cytometry tubes, incubated with chimeric mAb43 (106 cells/1 μL mAb43 stock at 1 mg/ml) on ice for 1 hr, and then PE-conjugated with anti-human IgG secondary antibody (106 cells/1 μL antibody stock at 1 mg/ml) for 1 hr on ice. Chimeric mAb20 was used as an isotype control.

Fluorescence activated cell sorting and confocal microscopy analysis. The labeled pancreatic cells were analyzed and sorted immediately on a MoFlo XDP cell sorter (Beckman Coulter) equipped with a 405 and 561 nm laser. Data were collected on forward scatter, side scatter, and 440 nm and 578 nm fluorescence channels. Cells gated on forward and side scatter yielded >1 million single-cell counting events. The sorted cells in R0 or R1 gate were deposited on the glass bottom of a microwell dish by a gentle centrifugation (1200 rpm, 1 min). After attachment to a matrigel (1:100) coated surface, cells were fixed with 4% paraformaldehyde for 20 min and subsequently permeabilized. Intracellular labeling was carried out in permeabilization buffer containing 2% BSA with chimeric mAb43, followed by anti-human-IgG-PE, anti-insulin APC, and anti-glucagon-Alexa Fluor 488. Following washing and nuclear DAPI counterstaining, immunofluorescence images were acquired using a Zeiss LSM 700 as described above.

Western blot analysis of mAb biodistribution in mice. 10 to 11-week-old male C57BL/6 mice were given chimeric mAb43 or chimeric mAb20 at a dose of 5 mg/kg through intravenous or intraperitoneal administration. One to six days post-injection, mice were euthanized, and tissues from various organs were excised, dried by a brief spin on a strainer, weighed, homogenized in PBS with DNase and protease inhibitors. The tissue suspension was dissolved in 4×SDS-PAGE sampling buffer at a concentration of 50 mg/ml. Chimeric mAb43 or chimeric mAb20 in each tissue was detected by anti-human-IgG immunoblotting and quantified using serial dilutions of a human IgG standard on the same blot. The tissue uptake was corrected for tissue weight and total administered mAb dose; the amount of antibody retained was calculated as a percentage of injected mAb per gram of each tissue collected (% mAb injected/g).

Preparation of flattened wholemount pancreas. 10 to 11-week-old male/female C57BL/6 mice were given mAb43-mScarlet, mAb20-mScarlet, or PBS through intravenous administration at a dose of 5 mg/kg. One day after injection, mice were euthanized, and the whole pancreas was excised, placed between a pair of microscope slides, flattened by placing a heavy weight on top of the glass sandwich, and fixed with 4% PFA for 2 hr. The partially fixed pancreas was then removed from the glass sandwich and fixed for an additional 4 hours. Next, the fixed pancreas was transferred to saturated sucrose for about 48 hr, and then transferred to 100% glycerol overnight. The entire procedure from tissue flattening to optical clearing was performed in a cold room (8° C.) to minimize tissue degradation. For β-cell immunolabeling, the flattened and PFA-fixed pancreas was transferred to 1% Triton X-100 PBS plus 2% BSA overnight. Next, the pancreas was incubated with anti-insulin-APC (1:50) in 0.1% Triton X-100 with 0.2% BSA for 12 hours, washed, and subjected to optical clearing as described above. The cleared wholemount pancreas was placed between a microscope slide and a coverslip, and then flattened again using a heavy weight while sealing the coverslip with fluorogel. A pair of 10-week-old male/female MIP-GFP mice was given mAb43-mScarlet at a triple dose (15 mg/kg) through intraperitoneal administration. Three days post-injection, pancreata were excised and subjected to PFA-fixation and optical clearing, as described above.

Imaging wholemount pancreas and data analysis. Images of the wholemount pancreas were acquired on an ImageXpress Micro high-content analysis system with a 4×/0.2 PlanApo objective lens. Laser-autofocus controlled by MetaXpress software was fixed on the glass surface (20 mm W.D.), and a maximum projection from 3D-reconstruction of 17×10 μm Z-stacks (˜0.2 mm tissue thickness) yielded a 2D-projection image with 16-bit planar resolution, 3-log intensity range and 3-colors each position from a Lumencor SOLA solid-state fluorescence light source using the GFP (488 nm), Rhodamine (585 nm) and Cy5 (692 nm) filter sets for GFP, mAb43-mScarlet and insulin-APC fluorescence, respectively. Transmission light scanning was recorded simultaneously to produce a bright field image. Exposure times for each fluorescence channel (100-200 ms) were selected to have just enough exposure to show autofluorescence of pancreata from mice given PBS or mAb20-mScarlet injection. A tiled scan of the wholemount pancreas on a motorized stage generated grid of images, which were combined using the Fiji stitching plugin to generate a merged image. The flattened pancreas preparation and optical clearing gave a uniform autofluorescence background. A single background fluorescence level was measured for each fluorescence channel, subtracted numerically across the entire image, and then displayed by ImageJ without further modification. Mander's overlap coefficients were computed across the whole pancreas using all pixels above auto-thresholds for GFP and mScarlet fluorescence without background correction.

Mouse pancreatic islet preparation and imaging. Mouse pancreas was perfused by 5 ml of pre-chilled collagenase P (1 mg/ml) by cannulating the bile duct attached to the duodenum at the papilla while the bile duct bundle near the liver was closed by suturing (44). The fully inflated pancreas was excised, digested at 37° C. for 7 min, washed in G-solution (HBSS plus 0.35 g/L NaHCO3 and 1% BSA), filtrated through a mesh, and then pelleted at 1200 rpm for 2 min. The pellet was resuspended in 15 ml Histopaque 1100 (45), and islets were separated from tissue debris by centrifugation at 1200 rpm for 20 min. The upper layer was collected, diluted with 25 ml G-solution, and then islets were pelleted at 1500 rpm for 4 min with 2× wash. The pellet was resuspended in islet culture medium (RPMI 1640 plus 2 mM L-glutamine, 10% FBS, 100 U/ml penicillin, and 100 μg/ml streptomycin). Healthy islets were picked into fresh culture medium supplemented with 20 mM glucose in a glass bottom microwell dish. mAb43-mScalet or mAb20-mScalet was added to the islet culture medium to a final concentration of 0.01 mg/ml, incubated for 2 hr in a 37° C. CO2 incubator, washed once with HBSS buffer, and then loaded into a glass sandwich (˜0.4 mm spacing) used for wholemount pancreas imaging. Islet imagers were acquired at room temperature on an ImageXpress Micro high-content analysis system using the same settings as for wholemount pancreas imaging, as described above.

Statistical analysis. All values are expressed as the mean±standard error of the mean. Two-tailed Student's t test is used to compare groups. Significance indicated in the figures is denoted *; P<0.01.

### Example 2: Induction of Anti-TMD Antibodies and Biochemical Characterization

Lymphocytes responsible for the production of antibodies to highly conserved epitopes of ZnT8 may be eliminated during the development of self-tolerance that prevents lymphocytes from attacking self-antigens. To overcome this impediment, two different immunization strategies were used to elicit antibody responses to human ZnT8: 1) deleting the ZnT8 gene to avoid negative selection in immunologically intact mice; and 2) stimulating autoreactivity to ZnT8 in immunologically comprised mice with defective immune tolerance. Accordingly, the present inventors immunized ZnT8-KO mice and non-obese diabetic (NOD) female mice that are prone to developing spontaneous autoimmune diabetes. To preserve the native folding of ZnT8 antigen once injected into the blood circulation, the present inventors developed a liposome-reconstituted ZnT8 formulation (14,16). ZnT8 is a two-modular protein consisting of a transmembrane domain (TMD) and a cytosolic C-terminal domain (CTD) (FIG. 1A). Since the native folding of the TMD requires the presence of the CTD, the mouse antibody response to the TMD was interrogated by comparative ELISAs against full-length ZnT8 (flZnT8) and its CTD. Both mouse strains showed robust anti-flZnT8 (TMD+CTD) and anti-CTD responses above the background levels of mice that received empty liposome injections as a control. The ZnT8-KO mice exhibited no difference in serum titrations against flZnT8 and the CTD, suggesting that all serum antibodies were directed to the CTD (FIG. 1C). By comparison, NOD mice exhibited a significantly higher serum reactivity toward flZnT8 at lower serum dilutions (FIG. 1D), suggesting the presence of anti-TMD reactivity in proteoliposome-injected NOD mice, in addition to CTD reactivity. Next, the present inventors generated hybridoma cells from immunized ZnT8-KO and NOD mice. All mAbs derived from ZnT8-KO mice targeted the intracellular CTD portion of ZnT8. Similarly, mAbs derived from NOD mice predominantly recognized the CTD. Nevertheless, the present inventors identified an anti-TMD mAb (mAb43) that was exclusively reactive to flZnT8 (TMD+CTD) with no detectable reactivity to the CTD (FIG. 1E-1F). Reconstitution of detergent-solubilized human ZnT8 into proteoliposomes increased mAb43 reactivity by 6.29-fold, demonstrating a preferential recognition of the natively folded TMD conformation in the membrane (FIG. 1G). A validated anti-CTD mAb20 was used as a binding control (17). No difference was observed in mAb20 reactivities to three different antigen formats: detergent-solubilized ZnT8, CTD, and liposome-reconstituted ZnT8 (FIG. 1E-1G). This mAb20 binding profile is consistent with CTD as an independently folded soluble domain (18). mAb43 and mAb20 titrations to ZnT8 proteoliposomes yielded binding affinities at 0.42±0.05 and 0.57±0.07 nM, respectively.

### Example 3: Cell Surface Binding and Specificity

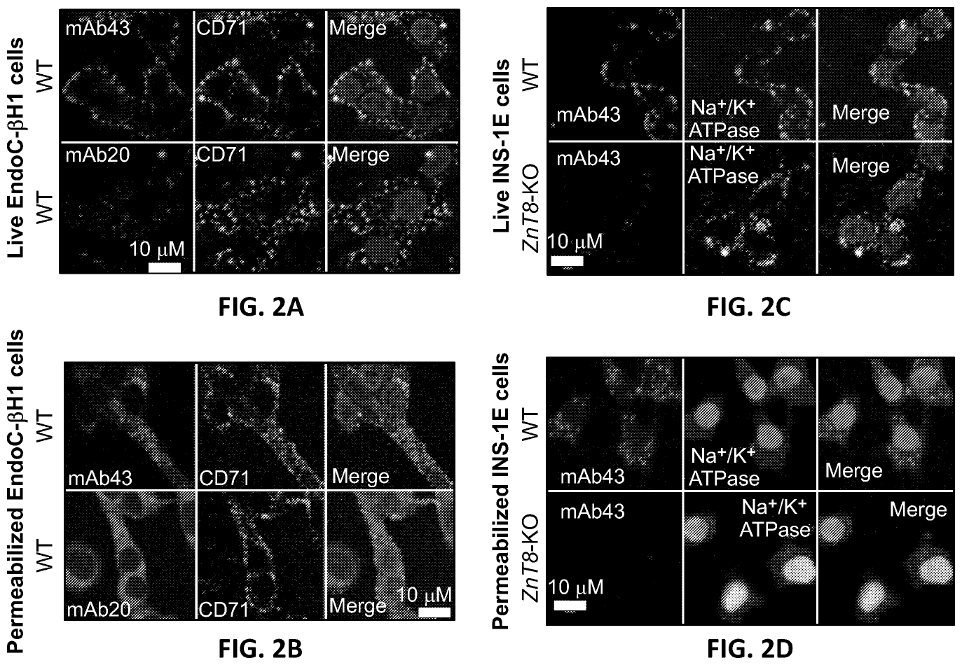

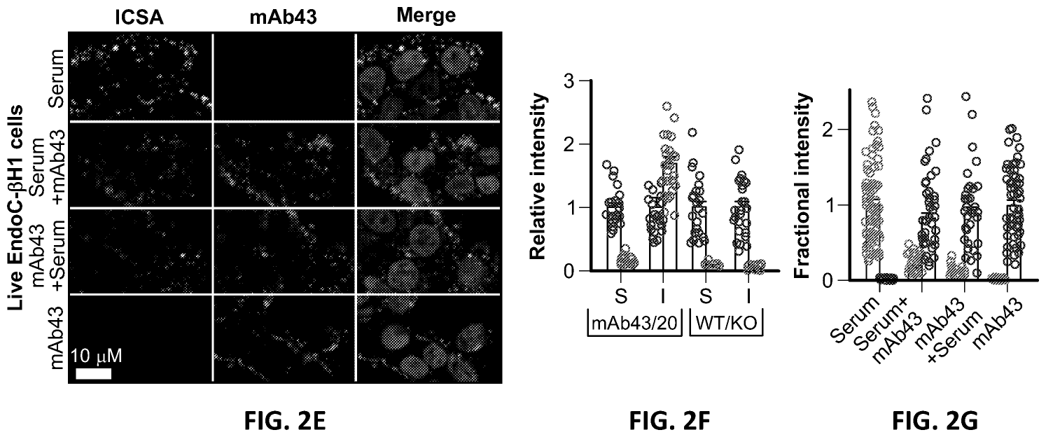

To determine whether the observed anti-TMD reactivity of mAb43 was directed to the extracellular surface of the TMD, immunofluorescence (IF) labeling of live human β-cells (EndoC-βH1) by mAb43, mAb20 and an antibody against the abundant cell surface marker CD71 was compared. All experiments were performed at 8° C. to arrest antibody endocytosis and in the presence of 20 mM glucose to stimulate ZnT8 surfacing (11). mAb43 and anti-CD71 yielded strong IF punctation on the cell surface whereas mAb20 did not produce a detectable signal (FIG. 2A). On the other hand, both mAb20 and mAb43 strongly labeled permeabilized EndoC-βH1 cells, due to their recognitions of the cytosolic CTD and luminal TMD epitope, respectively (FIG. 2B). The present inventors further examined mAb43 cross-reactivity to a rat β-cell line (INS-1E) in comparison with a rodent-reactive antibody against the abundant cell surface marker Na+/K+ ATPase. mAb43 and anti-Na+/K+ ATPase yielded strong IF punctation on the cell surface of live INS-1E cells (FIG. 2C). By comparison, immunolabeling of permeabilized INS-1E cells revealed vesicular and nuclear labeling by mAb43 and Na+/K+ ATPase antibody, respectively (FIG. 2D). Na+/K+ ATPase has previously been reported to be localized to the nuclear membrane in addition to the cell surface (19). Finally, CRISPR/Cas9-mediated ZnT8-knockout in INS-1E cells abolished IF-labeling of mAb43 on the cell surface as well as in intracellular vesicles, validating ZnT8 specificity in rodent β-cells (FIG. 2C-2D). Quantifying the differences in mAb43 or mAb20 IF-labeling of EndoC-bH1 cells, and mAb43 IF-labeling of wild type or ZnT8-KO INS-1E cells further validated specific mAb43 immunolabeling of cell surface ZnT8 (FIG. 2F). Lastly, competitive ZnT8 binding by mouse mAb43 and a human serum that was previously tested positive for ZnT8ecA was examined. Exposing live EndoC-bH1 cells to either mouse mAb43 or the human serum yielded strong mouse or human IgG punctation on the cell surface. By comparison, exposing live EndoC-βH1 cells to both mouse mAb43 and human serum predominantly yielded mouse IgG punctation, regardless of serum or mAb43 pre-blocking (FIG. 2E). Imaging quantification indicated that mAb43 displaced over 80% of serum IgG punctation on the cell surface (FIG. 2G). This finding indicates that polyclonal serum ZnT8ecAs from a diabetic patient are pronominally directed to a cell surface ZnT8 epitope shared by mAb43.

### Example 4: Epitope Mapping and Conformation Specificity

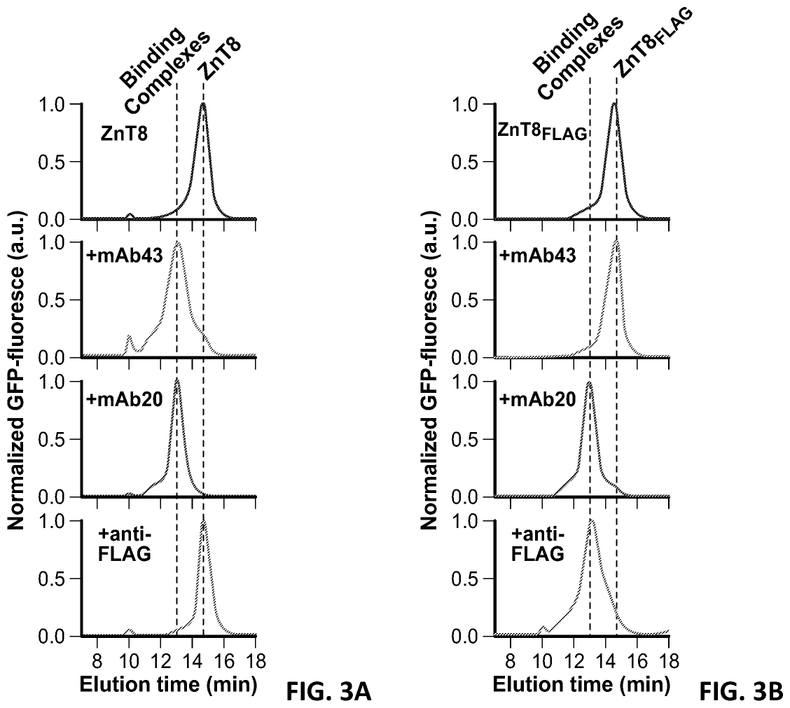

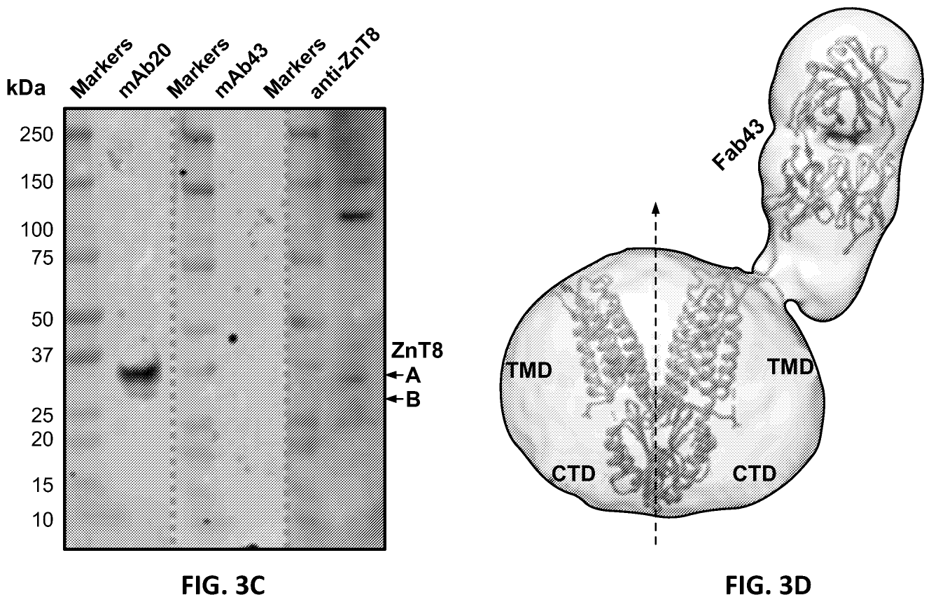

To map the mAb43 epitope to ZnT8 extracellular loops (ECLs), the present inventors inserted a FLAG-octapeptide into individual ECLs to perturb their local conformation, and then compared mAb43 binding to native ZnT8 and ZnT8FLAG. Among nine insertion constructs, only an ECL-2 insertion resulted in ZnT8FLAG expression in INS-1E cells (11). An enhanced green fluorescence protein (GFP) was appended to the ZnT8 C-terminus to monitor the formation of a binary mAb-ZnT8 complex by fluorescence size-exclusion HPLC. mAb43 binding shifted the ZnT8-GFP peak leftward, indicating the formation of a stable mAb43-ZnT8-GFP complex (FIG. 3A). The FLAG-tag abolished mAb43 binding to ZnT8FLAG-GFP, but added anti-FLAG binding that formed a stable anti-FLAG-ZnT8FLAG-GFP complex (FIG. 3B). The FLAG-tag neither altered the monodispersed profile of ZnT8FLAG-GFP, nor affected the formation of a mAb20-CTD complex (FIG. 3B). Hence, mAb43 and FLAG antibody directly competed for ECL-2 on the TMD surface of a natively folded ZnT8. Moreover, mAb43 was not reactive to SDS-denatured ZnT8 on immunoblots, despite the presence of an unaltered ECL-2 loop (FIG. 3C). This finding further demonstrated the conformation specificity of mAb43. By comparison, mAb20 detected two SDS-denatured ZnT8 splice variants in the lysate of EndoC-βH1 cells (4,17), while an anti-peptide ZnT8 antibody detected denatured ZnT8 with high non-specific reactivities (FIG. 3C). Lastly, negative-stain electron microscopy (EM) single-particle analysis was used to visualize the binding complex of an antigen-binding fragment of mAb43 (Fab43) with detergent-solubilized ZnT8. Delipidated ZnT8 was not able to form a stable Fab43-ZnT8 complex to survive the EM grid preparation. Nevertheless, a Fab43-ZnT8 complex was captured using minimally delipidated ZnT8, and only one Fab43 molecule was found in complex with a ZnT8 homodimer (FIG. 3D). The point of Fab43 attachment to the ZnT8 homodimer density was ˜18o off the two-fold homodimer axis, in alignment with a splayed TMD. This mode of Fab43 binding is clearly distinct from the docking of Fab20 to the CTD at the two-fold axis in previously reported Fab20-ZnT8 complexes (12,17). Since the two ZnT8 protomers in a ZnT8 homodimer adopt distinct conformations (20), Fab43 appeared to recognize either an outward- or inward-facing conformation. Taken together, the biochemical data indicate that mAb43 forms a stable complex with ZnT8 through conformation-specific binding to ECL-2 loop.

### Example 5: Specificity for Mouse Islets and β-Cells

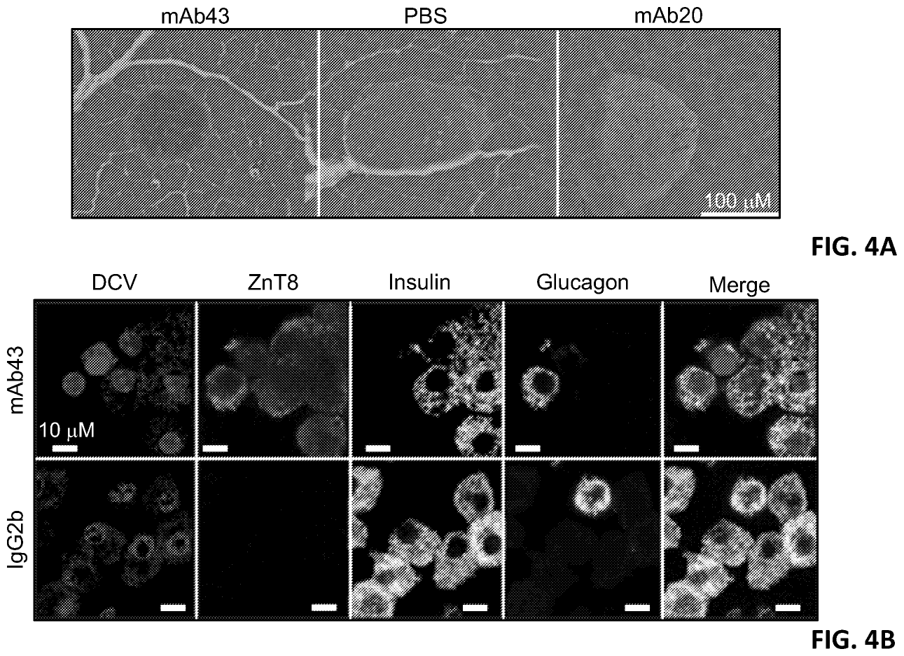

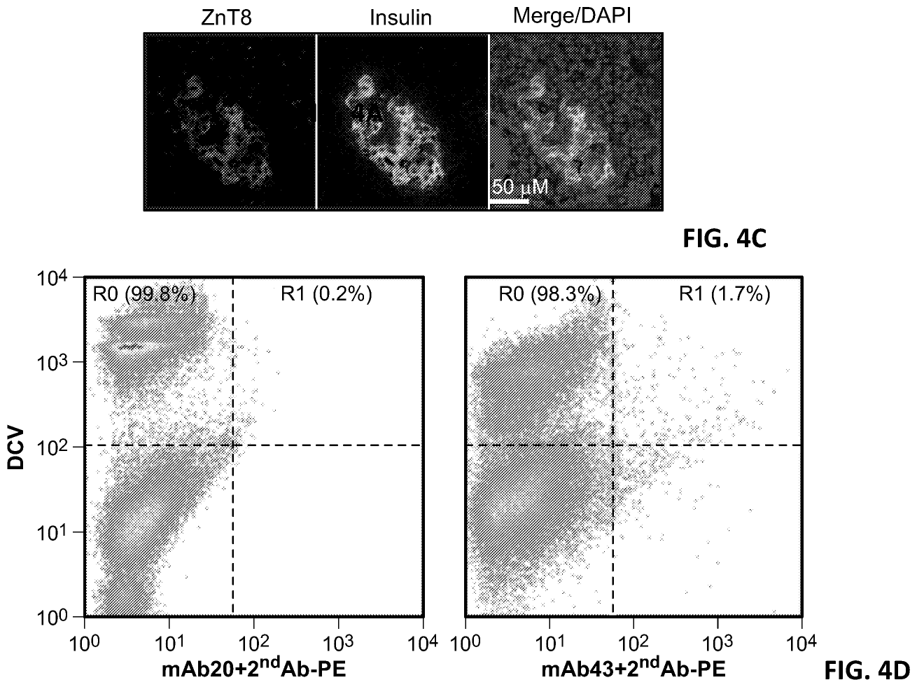

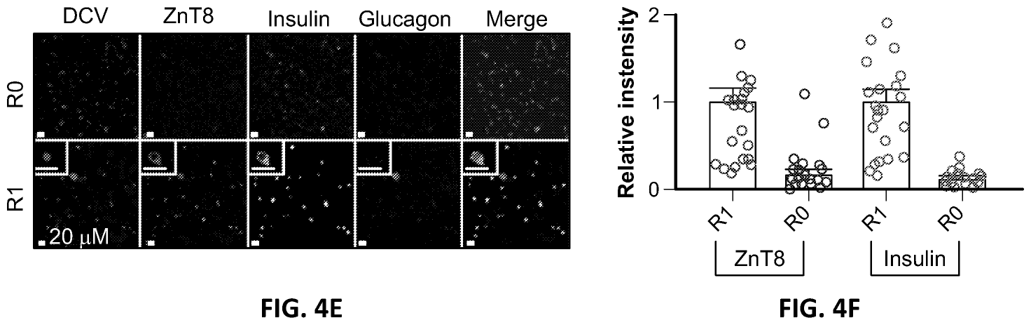

mAb43 specificity was examined ex vivo in paraffin-embedded mouse pancreas sections. mAb43 labeling and diaminobenzidine immunohistochemistry revealed specific localization of mAb43 binding to islets of Langerhans. By comparison, mAb20 did not immunolabel islets due to a lack of cross-reactivity to mouse ZnT8 (FIG. 4A). Co-immunolabeling of enzymatically dispersed and detergent permeabilized mouse islet cells with anti-insulin, anti-glucagon and mAb43 showed that mAb43 recognized both a- and b-cells while an isotype (IgG2b) control did not yield a detectable IF-signal (FIG. 4B). Co-immunolabeling of human pancreatic cryosections from two different patients with T2D revealed co-localization of anti-insulin and mAb43 IF-signals, demonstrating the specificity of mAb43 for human islets (FIG. 4C). For some islets, a halo of cells without brown stain was evident; those cells are likely α- and δ-cells that are typically localized in the periphery of normal mouse islets. Next, fluorescence-activated cell sorting was used to examine mAb43 labeling of β-cells in mixed cell populations of enzymatically dispersed mouse pancreata. mAb43-labeled cells were detected using a phycoerythrin (PE) conjugated secondary antibody while intact islet cells were gated against large cell debris and granular vesicles based on positive staining by a cell permeable DNA dye, DyeCycle Violet (DCV). Forward and side scatter restrictions were applied to gate single-cell events. Only a small fraction (1.7%) of the pancreatic dispersion fell into the DCV(+)/mAb43-PE(+) quadrant (FIG. 4D). This low percentage is consistent with the pancreatic β-cell population that comprises less than 2% of the overall pancreatic mass. The sorted cells were grown on a matrigel-coated glass surface, then fixed, permeabilized, and subjected to triple IF-staining for ZnT8, insulin and glucagon. All sorted DCV(+)/mAb43-PE(+) cells were positive for both ZnT8 and insulin, but negative for glucagon (FIG. 4E). By comparison, most sorted DCV(+)/mAb43-PE(−) cells were negative for ZnT8, insulin and glucagon. Quantification of insulin and ZnT8 immunolabeling revealed a clear enrichment of b-cells correlated with elevated mAb43 IF-intensity (FIG. 4F). Thus, flow sorting of mAb43-labeled cells allowed separation of β-cells from the acinar and ductal tissue that makes up the bulk of pancreas mass (98.3%). The mouse-reactivity of mAb43 indicates that mAb43 arose from NOD autoimmunity against a ZnT8 self-epitope. The mAb43 specificity for primary mouse β-cells is consistent with the highly selective nature of NOD autoimmunity against β-cells, while the remainder of islet cells is autoimmune tolerated.

### Example 6: Glucose-Stimulated ZnT8-mAb43 Uptake

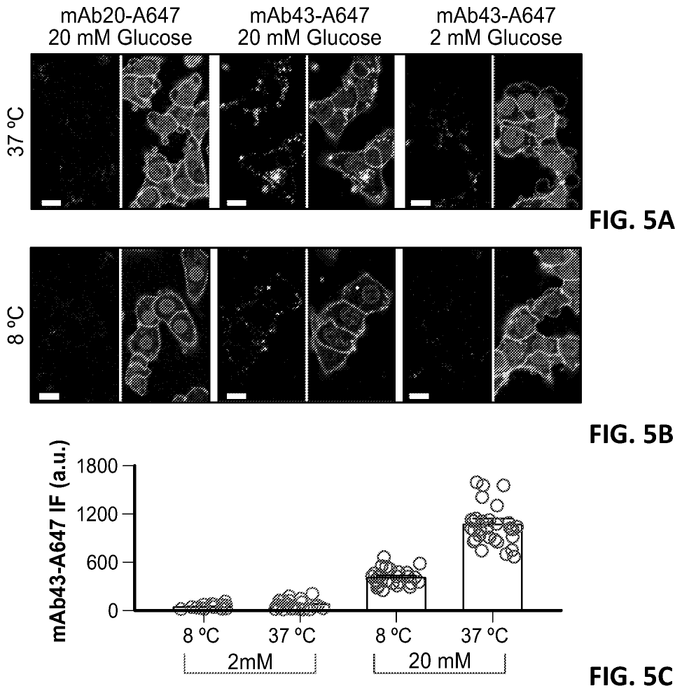

To track cell-surface capture of mAb43 and the ensuing ZnT8-mediated mAb43 endocytosis, a fluorescent A647 secondary antibody to label mAb20 and mAb43, and a CellMask green stain were used to demarcate the cell boundary. Live EndoC-βH1 cells were monitored for antibody surface binding and internalization. mAb43-A647 was rapidly internalized at 37° C. whereas mAb20-A647 exposure yielded no detectable signal (FIG. 5A). When EndoC-βH1 cells were chilled at 8° C., mAb43-A647 endocytosis arrested, but cell surface binding of mab43-A647 persisted (FIG. 5B). Importantly, lowering glucose concentration from 20 to 2 mM markedly reduced both mAb43 cell-surface binding at 8° C. and mAb43-A647 uptake at 37° C. (FIG. 5A-5B). Imaging quantification suggested that glucose stimulation (20 mM) increased total mAb43-A647 IF-labeling by 22.1- and 15.0-fold at 37° C. and 8° C., respectively (FIG. 5C). The difference in mAb43-A647 IF signals between 37° C. and 8° C. approximated to the net mAb43-A647 uptake. Glucose stimulation increased ZnT8-mediated mAb43 uptake by 30.9-fold (FIG. 5C). Glucose-dependent mAb43 capture and internalization were also observed using a fusion of mAb43 with a monomeric red fluorescent protein, mScarlet.

### Example 7: In Vivo mAb43 Biodistribution in Mice

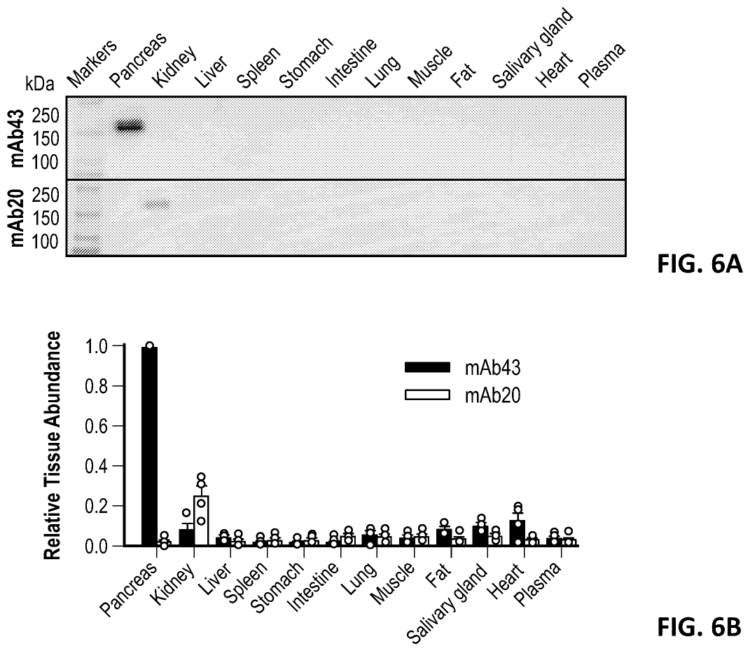

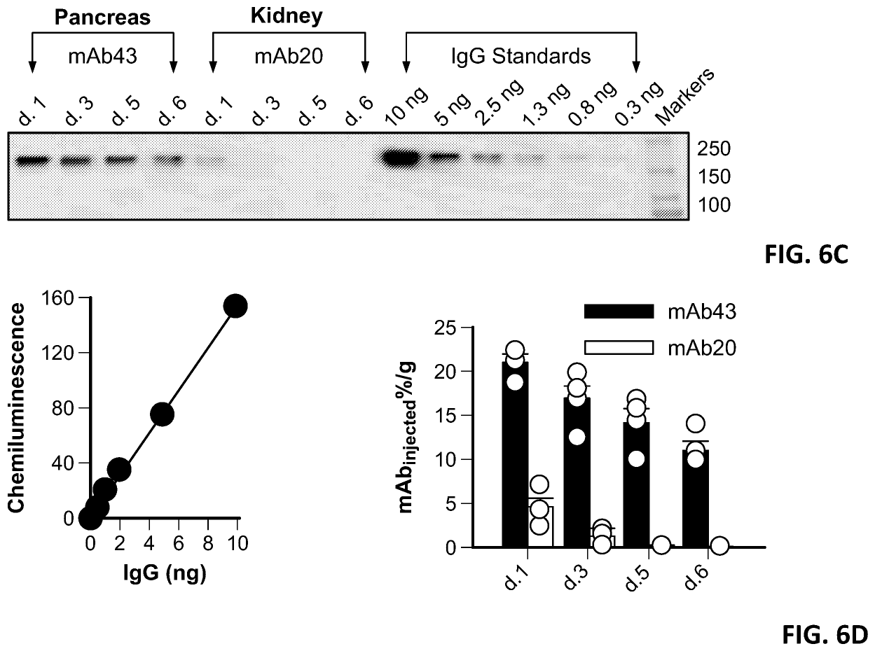

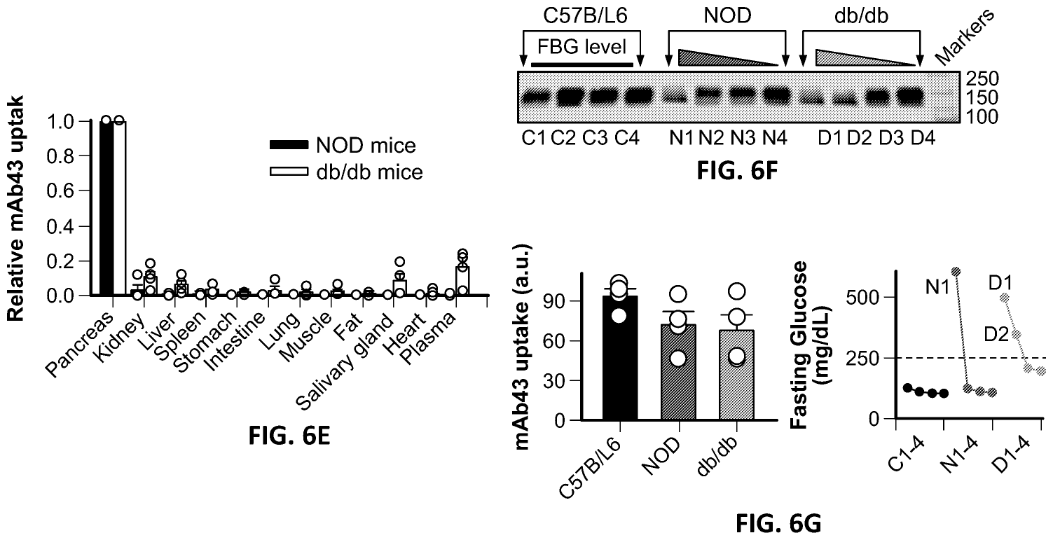

To characterize in vivo mAb43 uptake in mice, a mouse-Fab/human-Fc chimeric mAb43 was generated, injected four male C57BL/6 mice (C1-4) at a low dose of 5 mg/kg, and then used anti-human-IgG immunoblotting to detect the chimeric mAb43 in a panel of excised organs. C1-C3 received mAb43 intravenously and C4 intraperitoneally. Circulating mAb43 in the plasma was rapidly eliminated within a day (FIG. 6A), in agreement with the mouse pharmacokinetic model of target-mediated antibody clearance for low-dose administration. From 1 to 6 days post-injection, mAb43 was detected predominantly in the pancreas, and its biodistribution profile remained unchanged regardless of administration route (FIG. 6B). The pancreas-to-serum ratio of mAb43 ranged from 24.6 to 66.2. Control experiments with a mouse/human chimeric mAb20 yielded no detectable signal in the pancreas by 3 days post injection (FIG. 6A-6C). By comparison, the half-life of mAb43 in the pancreas was approximately a week, with an initial pancreas concentration of 21.1±0.9% mAb_{injected}/g 1-day post injection, tapering down to 14.3±1.5 and 11.1±1.0% mAb_{injected}/g at 5- and 6-day post injection, respectively (FIG. 6D). The pancreas-specific mAb43 biodistribution demonstrates the feasibility of targeting mAb43 to the pancreas through systemic administration. This finding, in conjunction with the ex vivo mAb43 specificity for islets (FIGS. 4A-4F), further suggests that mAb43 is specifically directed to pancreatic islets.

Next, mAb43 biodistribution was examined in mouse model of T1D and T2D. Four NOD females (N1-N4) and four db/db males (D1-D4), both at 18 weeks of age, were given a single mAb43 dose of 5 mg/kg by intraperitoneal injection, then the tissue uptake of mAb43 was measured 48-hour post-injection. At 18 weeks of age, the lymphocytes infiltration in pancreatic islets of NOD females are well established, while overt obesity is developed in db/db males. Both mouse strains exhibited biodistribution profiles similar to that of C57BL/6 with mAb43 predominately accumulated in the pancreas (FIG. 6E). The levels of pancreatic mAb43 uptake were compared among individual mice of different stains with different fasting blood glucose (FBG) levels ranging from normoglycemia to hyperglycemia (FIG. 6F). On average, C57BL/6 mice had a modestly higher mAb43 uptake than the NOD and db/db mice, respectively (FIG. 6G). One NOD and two db/db mice become diabetic (FBG>250 mg/dL), and these mice exhibited significant reduction of pancreatic mAb43 uptake.

### Example 8: Targeted Delivery of mScarlet to Pancreatic Islets

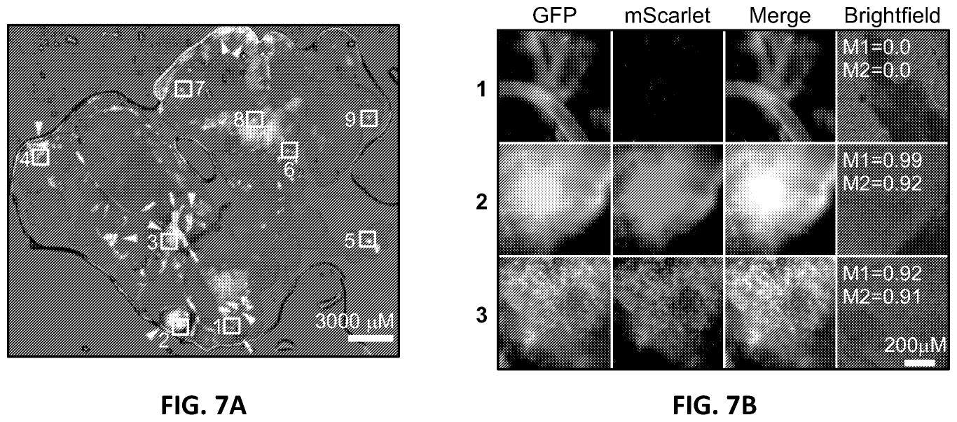

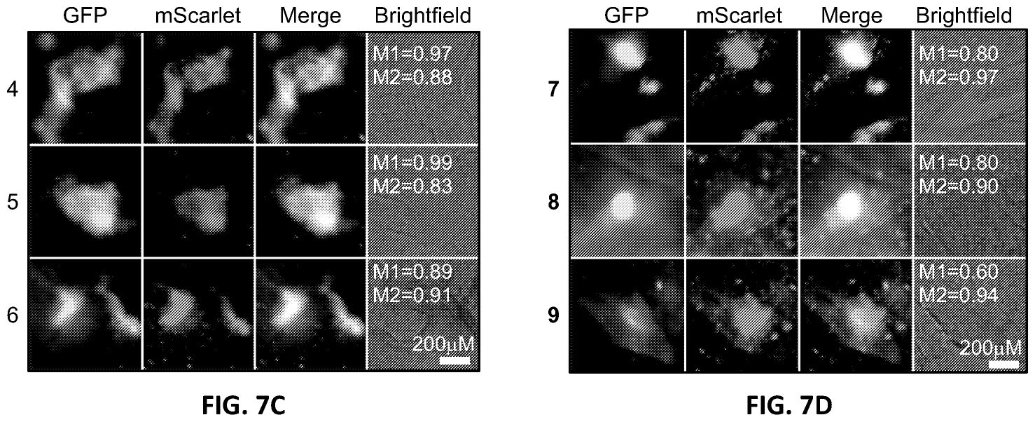

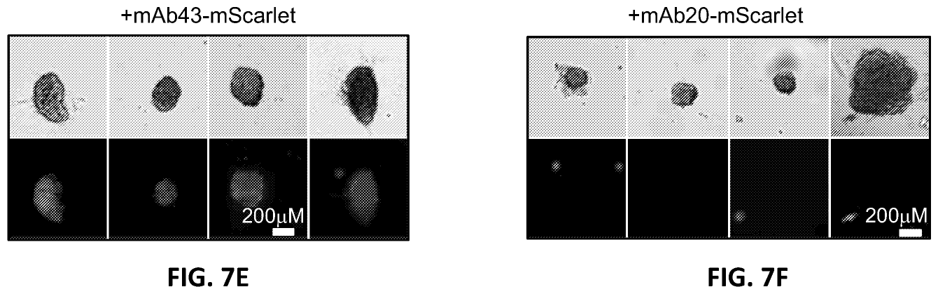

To evaluate the feasibility of mAb43 for in vivo delivery of imaging payloads, the present inventors injected C57BL/6 mice with mAb43-mScarlet, mAb20-mScarlet or PBS control, and then performed wholemount pancreas imaging to detect mScarlet uptake in excised pancreata. Only mAb43-mScarlet injection resulted in distinctive mScarlet puncta across the whole pancreas. Anti-insulin-APC immunolabelling of β-cells in detergent-permeabilized pancreata yielded a similar distribution of APC puncta, but the detergent treatment ablated mScarlet puncta due to the loss of intracellularly trapped mScarlet. To directly evaluate islet-homing of mScarlet, the present inventors used GFP-tagged β-cells in a transgenic MIP-GFP mouse that received a mAb43-mScarlet injection (25). Wholemount pancreas imaging revealed a high degree of global colocalization between GFP and mScarlet with Mander's overlap coefficients of 0.93 and 0.79 for the fraction of mScarlet overlapping GFP, and for GFP overlapping mScarlet, respectively (FIG. 7A). The 21% unmatched GFP signal was largely attributed to erythrocyte GFP autofluorescence in pancreas arteries and their branches, where the mScarlet signal was completely absence (FIG. 7B). In contrast, GFP-mScarlet co-occurrence was nearly absolute in islet clusters that surrounded large blood vessels (FIG. 7B). Similar vasculature-associated islet clusters have been reported in the human pancreas (26). High-power magnification confirmed that individual GFP and mScarlet puncta co-localized (FIG. 7C). In some regions, minor mScarlet signals scattered without overlapping GFP signals (FIG. 7D); they were probably small j-cell clusters whose GFP signals were invisible when detergent was not used during tissue clearing (27). Finally, isolated mouse islets were examined for mScarlet uptake ex vivo. The sizes of individual islets were consistent with the sizes of mScarlet clusters revealed by wholemount pancreas imaging. mAb43-mScarlet exposure of isolated mouse islets resulted in intense mScarlet fluorescence, while exposure with mAb20-mScarlet resulted in no detectable uptake (FIG. 7E-7F). These findings further demonstrated the specificity of mAb43-mediated mScarlet uptake through ZnT8 binding on the j-cell surface.

The data indicate that the generation of mAb43 depends on self-tolerance breakdown in NOD mice where CD4+ autoreactive T cells to ZnT8 occur spontaneously, but they are weakly pathogenic. Accordingly, ZnT8-proteoliposome immunization is required to boost autoreactivity to ECLs. Deleting ZnT8 gene in ZnT8-KO mice is insufficient to induce antibodies against ECLs of human ZnT8, probably because the extracellular ZnT8 epitope is conserved across species in other homologs (ZnT1-10) in the ZnT protein family. In particular, a part of the ZnT signature sequence is located in ECL1. The resultant mAb43 is an autoantibody recognizing a cell-surface ZnT8 epitope with the hallmark in vivo islet specificity of T1D. The subnanomolar binding affinity of mAb43 is a rare occurrence in the spontaneous autoantibody repertoire of NOD mice. The mAb43-ZnT8 binding is distinctively conformation-specific. Multiple ECLs and their interactions are required to form a recognizable conformation to mAb43, because individual ECLs are too short to fold independently. Given a multi-loop mAb43 epitope on a limited extracellular surface area of ZnT8, the mAb43 epitope either in its entirety or at least a part of it should be shared by the polyclonal serum ZnT8ecA. Thus, the mAb43 binding can effectively protect the ZnT8 extracellular epitope against serum ZnT8ecA from patients with T1D. The IgD and IgM forms of mAb43 are BCRs of ZnT8-specific autoreactive B cells. mAb43 as a BCR could be used to investigate the molecular recognition and engagement of β-cells by autoreactive B-cells through the formation of a ZnT8-BCR(mAb43) centered immunological synapse.

The pancreas-specific biodistribution of mAb43 in conjunction of its islet-specific immunolabeling of pancreas sections suggest that systemically administrated mAb43 could be delivered specifically to pancreatic islets in vivo. Using mScarlet as a probe, wholemount pancreas imaging revealed regional mAb43-mScarlet enrichment in islet clusters on the periphery of the pancreas. These highly vascularized islets allow rapid insulin release into the circulation while the local GSIS activity is functionally coupled with ZnT8 surfacing and subsequent capture of the circulating mAb43. Pancreatic mAb43 uptake retains in diabetic mice of both T1D and T2D models, but the level of mAb43 uptake decreases, reflecting the loss of β-cells mass and/or function in diseased mice.

The in vivo islet-specificity of mAb43 is consistent with the islet-specific expression of ZnT8. Within the islet, ZnT8 is generally thought to be an intracellular protein expressed in all endocrine cell types. α-cells are the next most populous cell type after β-cells. While detergent-permeabilized α- and β-cells were immunolabeled by mAb43, only intact β-cells were FACS-enriched from the entire pancreatic cell population, suggesting that ZnT8 surfacing may be a β-cell specific function driven by GSIS. Antibodies recognizing specific markers on the β-cell surface could be used to target β-cells for the delivery of imaging agents or drugs that are toxic in non-islet tissues. Besides ZnT8 being targeted by mAb43 and another ZnT8 antibody (Ab31) directed to a peptide sequence of ECL2, sphingomyelin patches and NTPDase3 have been targeted as β-cell surface markers. Thus far, only mAb43 has demonstrated a pancreas-specific biodistribution profile that supports its utility for islet-homing of imaging payloads and antidiabetogenic drugs.

### Example 9: Use of mAb43 for In Vivo Imaging and Targeted Delivery of Antibody-Drug Conjugates

To evaluate the feasibility of mAb43 for in-vivo imaging and targeted delivery of antibody-drug conjugates, recombinant mAb43 with site-directed biotinylation at the C-terminus of the mAb43 heavy chain are generated. Biotin labeling is used to conjugate a fluorescent streptavidin as an imaging probe. Mouse pancreatic islet cells are labeled with mAb43-strepavidin, and sorted based on their cell surface IF-intensity and cellular zinc-sensitive fluorescence. The positively or negatively gated cells from FACS are subcultured, PFA-fixed and then IF-labeled with insulin and glycogen antibodies. A secondary flow cytometry analysis is expected to show that all mAb43-positive cells are insulin- or glucagon-positive, whereas mAb43-negative cells are insulin- or glucagon-negative. Further analysis may reveal a positive correlation of mAb43 and insulin positivity, indicating that the surfaced ZnT8 could be used as a biomarker for purification of the insulin-producing b-cells from a mixed population of islet cells. Some glucagon-producing α-cells may also be mAb43-positive, but the mAb43 IF-intensity are expected to be significantly lower than that of b-cells, and show no correlation with the cellular zinc content. Finally, biotinylated mAb43 is injected into mice, and tissue distribution of mAb43 by streptavidin-HPR is examined. Tissue histology is expected to reveal mAb43 accumulation in pancreatic islets, demonstrating in vivo targeted delivery of mAb43 to pancreatic islets.

### Example 10: Purification of Live Mature Stem Cell Derived Beta Cells (sBCs)

mAb43 is used to purify live mature sBCs from a heterogeneous cell mix. In an earlier experiment, mAb20 was used to sort C-peptide positive sBCs following PFA-fixation and permeabilization. Compared with mAb20, mAb43 has similar ZnT8 affinity and specificity, but the ZnT8 density on the cell surface is probably<5% of its intracellular density. Thus, bright dyes such as PE or APC may be for signal amplification. Recombinant mAb20/43 with site-directed fluorescence labeling are produced.

Studies are conducted to compare mAb43 to ENTPD3 (NTPDase3) and INS/Cpep to identify mature stem cell-derived beta cells (sBCs). hES, iPSC and T1D-iPSC sBC clusters that contain immature and mature sBCs are used.

Single cell suspensions of sBC clusters live are prepared and labeling efficiency using mAb is quantitated. mAb positive/negative populations are sorted, and then correlation with insulin/C-peptide expression is examined. These clusters/cells also contain a pInsulin-GFP reporter so mAb43-GFP correlation may be examined directly.

mAb43 is mouse IgG2b and control mAb20 is mouse IgG2a. The recombinant antibodies are switched to human IgG1-4. See SEQ ID NOS: 20-30.

TABLE 1
Sequence Identifier Number Table

| | |
|---|---|
| SEQ ID NO: 1 | NT sequence mAb43 heavy chain variable domain |
| SEQ ID NO: 2 | AA sequence mAb43 heavy chain variable domain |
| SEQ ID NO: 3 | AA sequence mAb43 heavy chain CDR1 |
| SEQ ID NO: 4 | AA sequence mAb43 heavy chain CDR2 |
| SEQ ID NO: 5 | AA sequence mAb43 heavy chain CDR3 |
| SEQ ID NO: 6 | NT sequence mAb43 light chain variable domain |
| SEQ ID NO: 7 | AA sequence mAb43 light chain variable domain |
| SEQ ID NO: 8 | AA sequence mAb43 light chain CDR1 |
| SEQ ID NO: 9 | AA sequence mAb43 light chain CDR2 |
| SEQ ID NO: 10 | AA sequence mAb43 light chain CDR3 |
| SEQ ID NO: 11 | AA sequence mAb20 heavy chain variable domain |
| SEQ ID NO: 12 | AA sequence mAb20 heavy chain CDR1 |
| SEQ ID NO: 13 | AA sequence mAb20 heavy chain CDR2 |
| SEQ ID NO: 14 | AA sequence mAb20 heavy chain CDR3 |
| SEQ ID NO: 15 | AA sequence mAb20 light chain variable domain |
| SEQ ID NO: 16 | AA sequence mAb20 light chain CDR1 |
| SEQ ID NO: 17 | AA sequence mAb20 light chain CDR2 |
| SEQ ID NO: 18 | AA sequence mAb20 light chain CDR3 |
| SEQ ID NO: 19 | AA sequence mAb43 heavy chain variable domain (1^{st} |
| | and last AA changed from SEQ ID NO: 2) |
| SEQ ID NO: 20 | AA sequence Light Chain: M43 (same for IgGs 1-4) |
| SEQ ID NO: 21 | AA sequence Heavy Chain: M43-hIgG1 |
| SEQ ID NO: 22 | AA sequence Heavy Chain: M43-hIgG2 |
| SEQ ID NO: 23 | AA sequence Heavy Chain: M43-hIgGw3 |
| SEQ ID NO: 24 | AA sequence Heavy Chain: M43-IgG4 |
| SEQ ID NO: 25 | AA sequence GS + 3xGGGGS linker |
| SEQ ID NO: 26 | AA sequence AVI tag sequence for site-directed |
| | biotinylation |
| SEQ ID NO: 27 | AA sequence Heavy Chain: hIgG1-GS-3XGGGGS- |
| | AVI |
| SEQ ID NO: 28 | AA sequence Heavy Chain: M43-hIgG2-GS- |
| | 3XGGGGS-AVI |
| SEQ ID NO: 29 | AA sequence Heavy Chain: M43-hIgG3-GS- |
| | 3XGGGGS-AVI |
| SEQ ID NO: 30 | AA sequence Heavy Chain: M43-hIgG4-GS- |
| | 3XGGGGS-AVI |
| SEQ ID NO: 31 | AA sequence of ZnT8 with ECLS1-3 featured |

### REFERENCES

1. Roep, B. O., Thomaidou, S., van Tienhoven, R., and Zaldumbide, A. (2021) Type 1 diabetes mellitus as a disease of the beta-cell (do not blame the immune system?). Nat Rev Endocrinol 17, 150-161.
2. Liston, A., Todd, J. A., and Lagou, V. (2017) Beta-Cell Fragility As a Common Underlying Risk Factor in Type 1 and Type 2 Diabetes. Trends Mol Med 23, 181-194.
3. Chimienti, F. (2013) Zinc, pancreatic islet cell function and diabetes: new insights into an old story. Nutr Res Rev 26, 1-11.
4. Merriman, C., and Fu, D. (2019) Down-regulation of the islet-specific zinc transporter-8 (ZnT8) protects human insulinoma cells against inflammatory stress. J Biol Chem 294, 16992-17006.
5. Dodson, G., and Steiner, D. (1998) The role of assembly in insulin's biosynthesis. Curr Opin Struct Biol. 8, 189-194.
6. Lemaire, K., Chimienti, F., and Schuit, F. (2012) Zinc transporters and their role in the pancreatic beta-cell. J Diabetes Investig 3, 202-211.
7. Segerstolpe, A., Palasantza, A., Eliasson, P., Andersson, E. M., Andreasson, A. C., Sun, X., Picelli, S., Sabirsh, A., Clausen, M., Bjursell, M. K., Smith, D. M., Kasper, M., Ammala, C., and Sandberg, R. (2016) Single-Cell Transcriptome Profiling of Human Pancreatic Islets in Health and Type 2 Diabetes. Cell Metab 24, 593-607.
8. Lukowiak, B., Vandewalle, B., Riachy, R., Kerr-Conte, J., Gmyr, V., Belaich, S., Lefebvre, J., and Pattou, F. (2001) Identification and purification of functional human beta-cells by a new specific zinc-fluorescent probe. J Histochem Cytochem 49, 519-528.
9. Lemaire, K., Ravier, M. A., Schraenen, A., Creemers, J. W., Van de Plas, R., Granvik, M., Van Lommel, L., Waelkens, E., Chimienti, F., Rutter, G. A., Gilon, P., in't Veld, P. A., and Schuit, F. C. (2009) Insulin crystallization depends on zinc transporter ZnT8 expression, but is not required for normal glucose homeostasis in mice. Proc Natl Acad Sci USA 106, 14872-14877.
10. Nicolson, T. J., Bellomo, E. A., Wijesekara, N., Loder, M. K., Baldwin, J. M., Gyulkhandanyan, A. V., Koshkin, V., Tarasov, A. I., Carzaniga, R., Kronenberger, K., Taneja, T. K., da Silva Xavier, G., Libert, S., Froguel, P., Scharfmann, R., Stetsyuk, V., Ravassard, P., Parker, H., Gribble, F. M., Reimann, F., Sladek, R., Hughes, S. J., Johnson, P. R., Masseboeuf, M., Burcelin, R., Baldwin, S. A., Liu, M., Lara-Lemus, R., Arvan, P., Schuit, F. C., Wheeler, M. B., Chimienti, F., and Rutter, G. A. (2009) Insulin storage and glucose homeostasis in mice null for the granule zinc transporter ZnT8 and studies of the type 2 diabetes-associated variants. Diabetes 58, 2070-2083.
11. Huang, Q., Merriman, C., Zhang, H., and Fu, D. (2017) Coupling of Insulin Secretion and Display of a Granule-resident Zinc Transporter ZnT8 on the Surface of Pancreatic Beta Cells. J Biol Chem 292, 4034-4043.
12. Gu, Y., Merriman, C., Guo, Z., Jia, X., Wenzlau, J., Li, H., Li, H., Rewers, M., Yu, L., and Fu, D. (2021) Novel autoantibodies to the beta-cell surface epitopes of ZnT8 in patients progressing to type-1 diabetes. J Autoimmun 122, 102677
13. Merriman, C., Huang, Q., Gu, W., Yu, L., and Fu, D. (2018) A subclass of serum anti-ZnT8 antibodies directed to the surface of live pancreatic beta-cells. J Biol Chem 293, 579-587.
14. Wan, H., Merriman, C., Atkinson, M. A., Wasserfall, C. H., McGrail, K. M., Liang, Y., Fu, D., and Dai, H. (2017) Proteoliposome-based full-length ZnT8 self-antigen for type 1 diabetes diagnosis on a plasmonic platform. Proc Natl Acad Sci USA 114, 10196-10201.
15. Eriksson, O., Korsgren, O., Selvaraju, R. K., Mollaret, M., de Boysson, Y., Chimienti, F., and Altai, M. (2018) Pancreatic imaging using an antibody fragment targeting the zinc transporter type 8: a direct comparison with radio-iodinated Exendin-4. Acta Diabetol 55, 49-57.
16. Merriman, C., Huang, Q., Rutter, G. A., and Fu, D. (2016) Lipid-tuned Zinc Transport Activity of Human ZnT8 Protein Correlates with Risk for Type-2 Diabetes. J Biol Chem 291, 26950-26957.
17. Merriman, C., Li, H., Li, H., and Fu, D. (2018) Highly specific monoclonal antibodies for allosteric inhibition and immunodetection of the human pancreatic zinc transporter ZnT8. J Biol Chem 293, 16206-16216.
18. Lu, M., and Fu, D. (2007) Structure of the zinc transporter YiiP. Science. 317, 1746-1748.
19. Garner, M. H. (2002) Na,K-ATPase in the nuclear envelope regulates Na+: K+ gradients in hepatocyte nuclei. J Membr Biol 187, 97-115.
20. Xue, J., Xie, T., Zeng, W., Jiang, Y., and Bai, X. C. (2020) Cryo-EM structures of human ZnT8 in both outward- and inward-facing conformations. Elife 9.
21. Bindels, D. S., Haarbosch, L., van Weeren, L., Postma, M., Wiese, K. E., Mastop, M., Aumonier, S., Gotthard, G., Royant, A., Hink, M. A., and Gadella, T. W., Jr. (2017) mScarlet: a bright monomeric red fluorescent protein for cellular imaging. Nat Methods 14, 53-56.
22. Saunders, D. C., Brissova, M., Phillips, N., Shrestha, S., Walker, J. T., Aramandla, R., Poffenberger, G., Flaherty, D. K., Weller, K. P., Pelletier, J., Cooper, T., Goff, M. T., Virostko, J., Shostak, A., Dean, E. D., Greiner, D. L., Shultz, L. D., Prasad, N., Levy, S. E., Carnahan, R. H., Dai, C., Sevigny, J., and Powers, A. C. (2018) Ectonucleoside Triphosphate Diphosphohydrolase-3 Antibody Targets Adult Human Pancreatic beta Cells for In Vitro and In Vivo Analysis. Cell Metab.
23. Noguchi, Y., Ozeki, K., and Akita, H. (2020) Pharmacokinetic prediction of an antibody in mice based on an in vitro cell-based approach using target receptor-expressing cells. Sci Rep 10, 16268.
24. Ovacik, M., and Lin, K. (2018) Tutorial on Monoclonal Antibody Pharmacokinetics and Its Considerations in Early Development. Clin Transl Sci 11, 540-552.
25. Hara, M., Wang, X., Kawamura, T., Bindokas, V. P., Dizon, R. F., Alcoser, S. Y., Magnuson, M. A., and Bell, G. I. (2003) Transgenic mice with green fluorescent protein-labeled pancreatic beta-cells. Am J Physiol Endocrinol Metab 284, E177-183.
26. Ionescu-Tirgoviste, C., Gagniuc, P. A., Gubceac, E., Mardare, L., Popescu, I., Dima, S., and Militaru, M. (2015) A 3D map of the islet routes throughout the healthy human pancreas. Sci Rep 5, 14634
27. Kim, A., Kilimnik, G., and Hara, M. (2010) In situ quantification of pancreatic beta-cell mass in mice. J Vis Exp.
28. Muratore, M., Santos, C., and Rorsman, P. (2021) The vascular architecture of the pancreatic islets: A homage to August Krogh. Comp Biochem Physiol A Mol Integr Physiol 252, 110846.
29. Hahn, M., Nord, C., Eriksson, M., Morini, F., Alanentalo, T., Korsgren, O., and Ahlgren, U. (2021) 3D imaging of human organs with micrometer resolution—applied to the endocrine pancreas. Commun Biol 4, 1063.
30. El-Gohary, Y., Sims-Lucas, S., Lath, N., Tulachan, S., Guo, P., Xiao, X., Welsh, C., Paredes, J., Wiersch, J., Prasadan, K., Shiota, C., and Gittes, G. K. (2012) Three-dimensional analysis of the islet vasculature. Anat Rec (Hoboken) 295, 1473-1481.
31. Bonner-Weir, S., and Orci, L. (1982) New perspectives on the microvasculature of the islets of Langerhans in the rat. Diabetes 31, 883-889.
32. Thomas, J. W., Kendall, P. L., and Mitchell, H. G. (2002) The natural autoantibody repertoire of nonobese diabetic mice is highly active. J Immunol 169, 6617-6624.
33. Arvan, P., Pietropaolo, M., Ostrov, D., and Rhodes, C. J. (2012) Islet autoantigens: structure, function, localization, and regulation. Cold Spring Harb Perspect Med 2.
34. Lernmark, A., Freedman, Z. R., Hofmann, C., Rubenstein, A. H., Steiner, D. F., Jackson, R. L., Winter, R. J., and Traisman, H. S. (1978) Islet-cell-surface antibodies in juvenile diabetes mellitus. N Engl J Med 299, 375-380.
35. Van De Winkel, M., Smets, G., Gepts, W., and Pipeleers, D. (1982) Islet cell surface antibodies from insulin-dependent diabetics bind specifically to pancreatic B cells. J Clin Invest 70, 41-49.
36. Tozzoli, R. (2020) Receptor autoimmunity: diagnostic and therapeutic implications. Auto Immun Highlights 11, 1.
37. Bloem, S. J., and Roep, B. O. (2017) The elusive role of B lymphocytes and islet autoantibodies in (human) type 1 diabetes. Diabetologia 60, 1185-1189.
38. Flannick, J., Thorleifsson, G., Beer, N. L., Jacobs, S. B., Grarup, N., Burtt, N. P., Mahajan, A., Fuchsberger, C., Atzmon, G., Benediktsson, R., Blangero, J., Bowden, D. W., Brandslund, I., Brosnan, J., Burslem, F., Chambers, J., Cho, Y. S., Christensen, C., Douglas, D. A., Duggirala, R., Dymek, Z., Farjoun, Y., Fennell, T., Fontanillas, P., Forsen, T., Gabriel, S., Glaser, B., Gudbjartsson, D. F., Hanis, C., Hansen, T., Hreidarsson, A. B., Hveem, K., Ingelsson, E., Isomaa, B., Johansson, S., Jorgensen, T., Jorgensen, M. E., Kathiresan, S., Kong, A., Kooner, J., Kravic, J., Laakso, M., Lee, J. Y., Lind, L., Lindgren, C. M., Linneberg, A., Masson, G., Meitinger, T., Mohlke, K. L., Molven, A., Morris, A. P., Potluri, S., Rauramaa, R., Ribel-Madsen, R., Richard, A. M., Rolph, T., Salomaa, V., Segre, A. V., Skarstrand, H., Steinthorsdottir, V., Stringham, H. M., Sulem, P., Tai, E. S., Teo, Y. Y., Teslovich, T., Thorsteinsdottir, U., Trimmer, J. K., Tuomi, T., Tuomilehto, J., Vaziri-Sani, F., Voight, B. F., Wilson, J. G., Boehnke, M., McCarthy, M. I., Njolstad, P. R., Pedersen, O., Groop, L., Cox, D. R., Stefansson, K., and Altshuler, D. (2014) Loss-of-function mutations in SLC30A8 protect against type 2 diabetes. Nat Genet 46, 357-363
39. Dwivedi, O. P., Lehtovirta, M., Hastoy, B., Chandra, V., Krentz, N. A. J., Kleiner, S., Jain, D., Richard, A. M., Abaitua, F., Beer, N. L., Grotz, A., Prasad, R. B., Hansson, O., Ahlqvist, E., Krus, U., Artner, I., Suoranta, A., Gomez, D., Baras, A., Champon, B., Payne, A. J., Moralli, D., Thomsen, S. K., Kramer, P., Spiliotis, I., Ramracheya, R., Chabosseau, P., Theodoulou, A., Cheung, R., van de Bunt, M., Flannick, J., Trombetta, M., Bonora, E., Wolheim, C. B., Sarelin, L., Bonadonna, R. C., Rorsman, P., Davies, B., Brosnan, J., McCarthy, M. I., Otonkoski, T., Lagerstedt, J. O., Rutter, G. A., Gromada, J., Gloyn, A. L., Tuomi, T., and Groop, L. (2019) Loss of ZnT8 function protects against diabetes by enhanced insulin secretion. Nat Genet 51, 1596-1606.
40. Kambe, T., Taylor, K. M., and Fu, D. (2021) Zinc transporters and their functional integration in mammalian cells. J Biol Chem, 100320.
41. Donath, M. Y., and Shoelson, S. E. (2011) Type 2 diabetes as an inflammatory disease. Nat Rev Immunol 11, 98-107.
42. Peterson, L. B., Bell, C. J. M., Howlett, S. K., Pekalski, M. L., Brady, K., Hinton, H., Sauter, D., Todd, J. A., Umana, P., Ast, O., Waldhauer, I., Freimoser-Grundschober, A., Moessner, E., Klein, C., Hosse, R. J., and Wicker, L. S. (2018) A long-lived IL-2 mutein that selectively activates and expands regulatory T cells as a therapy for autoimmune disease. J Autoimmun 95, 1-14.
43. Cieslak, M., Wojtczak, A., and Cieslak, M. (2015) Role of pro-inflammatory cytokines of pancreatic islets and prospects of elaboration of new methods for the diabetes treatment. Acta Biochim Pol 62, 15-21.
44. Stull, N. D., Breite, A., McCarthy, R., Tersey, S. A., and Mirmira, R. G. (2012) Mouse islet of Langerhans isolation using a combination of purified collagenase and neutral protease. J Vis Exp.
45. Corbin, K. L., West, H. L., Brodsky, S., Whitticar, N. B., Koch, W. J., and Nunemaker, C. S. (2021) A Practical Guide to Rodent Islet Isolation and Assessment Revisited. Biol Proced Online 23, 7.

1. An antibody or antigen-binding fragment thereof that specifically binds to three extracellular loops of the transmembrane domain of Zinc Transporter-8 (ZnT8), wherein the antibody or antigen-binding fragment thereof comprises:
(a) heavy chain complementarity determining regions (CDRs) 1, 2 and 3 comprising SEQ ID NOs: 3-5, respectively; and
(b) light chain CDRs 1, 2 and 3 comprising SEQ ID NOs: 8-10, respectively.

2. The antibody or antigen-binding fragment thereof of claim 1, wherein the three extracellular loops of ZnT8 comprise amino acids 95-99, 169-175 and 242-245 of SEQ ID NO: 31.

3. (canceled)

4. (canceled)

5. The antibody or antigen-binding fragment thereof of claim 1, wherein the antibody or antigen-binding fragment thereof comprises:
(a) a heavy chain variable region sequence having at least 90% sequence identity to SEQ ID NO: 2 or SEQ ID NO: 19; and
(b) a light chain variable region sequence having at least 90% sequence identity to SEQ ID NO: 7.

6. (canceled)

7. (canceled)

8. The antibody or antigen-binding fragment thereof of claim 1, wherein the fragment comprises a Fab, Fab′, F(ab′)₂, Fab¹-SH, Fv, diabody, linear antibody or single-chain variable fragment (scFv).

9. (canceled)

10. (canceled)

11. The antibody or antigen-binding fragment of claim 1, wherein the antibody or antigen-binding fragment thereof is conjugated to a therapeutic agent.

12. The antibody or antigen-binding fragment of claim 1, wherein the antibody or antigen-binding fragment thereof is conjugated to an imaging agent.

13. A pharmaceutical composition comprising a therapeutically effective amount of the antibody or antigen-binding fragment thereof of claim 1.

14. A nucleic acid molecule encoding the antibody or antigen-binding fragment of claim 1.

15. A vector comprising the nucleic acid of claim 14.

16. A host cell comprising the vector of claim 15.

17. A method for producing an antibody drug-conjugate that specifically binds three extracellular loops of the transmembrane domain of ZnT8, the method comprising:
(a) culturing the host cell of claim 16 under conditions suitable for production of the antibody; and
(b) conjugating the antibody to a therapeutic agent.

18. A method for producing an antibody imaging agent-conjugate that specifically binds three extracellular loops of the transmembrane domain of ZnT8, the method comprising:
(a) culturing the host cell of claim 16 under conditions suitable for production of the antibody; and
(b) conjugating the antibody to an imaging agent.

19. A method for treating a disease or condition associated with ZnT8 in a subject, the method comprising administering to the subject the antibody or antigen-binding fragment thereof of claim 1 or the pharmaceutical composition of claim 13, wherein the disease or condition comprises type 1 or type 2 diabetes.

20. (canceled)

21. A method for detecting pancreatic beta cells in vivo, the method comprising administering the antibody or antigen-binding fragment thereof of claim 12 to a subject and detecting the imaging agent conjugated to the antibody or antigen-binding fragment thereof.

22. The method of claim 21, wherein the detecting step comprises positron emission tomography (PET), single-photon emission computed tomography (SPECT)/CT imaging, nuclear magnetic resonance (NMR) spectroscopy or near-infrared (NIR) optical imaging.

23. (canceled)

24. (canceled)

25. The method of claim 21, wherein the imaging agent is a radiometal and the detecting step comprises PET, and wherein the radiometal is selected from the group consisting of ⁶⁴Cu, ⁶⁷Cu, ⁶⁸Ga, ⁶⁰Ga, ⁸⁹Zr, ⁸⁶Y, and ^{94m}Tc.

26. (canceled)

27. (canceled)

28. The method of claim 21, wherein the imaging agent is a radiometal and the detecting step comprises SPECT, and wherein the radiometal is selected from the group consisting of ¹¹¹In, ⁶⁷Ga, ^{99m}Tc, and ¹⁷⁷Lu.

29. (canceled)

30. A single-chain variable fragment comprising (scFv) or antigen-binding fragment thereof that binds to three extracellular loops of the transmembrane domain of ZnT8 comprising (a) a heavy chain variable region sequence of SEQ ID NO: 2 or SEQ ID NO: 19; and (b) a light chain variable region sequence of SEQ ID NO: 7.

31. (canceled)

32. The scFv of claim 30, wherein the scfv is conjugated to an imaging agent, wherein the imaging agent is a radiometal selected from the group consisting of ⁶⁴Cu, ⁶⁷Cu, ⁶⁸Ga, ⁶⁰Ga, ⁸⁹Zr, ⁸⁶Y, ^{94m}Tc, ¹¹¹In, ⁶⁷Ga, ^{99m}Tc, and ¹⁷⁷Lu.

33. (canceled)

34. (canceled)

35. An antibody or antigen-binding fragment thereof comprising (a) a light chain comprising SEQ ID NO: 20; and (b) a heavy chain comprising one of SEQ ID NOS: 21-24 or 27-30.

36. (canceled)


SEQUENCE LISTING (31 sequences)

SEQ ID NO: 1 - DNA, 351 bp, synthetic construct
  misc_feature 1..351: note = mAb43 heavy chain variable domain
  misc_feature 91..106: note = CDR1
  misc_feature 148..198: note = CDR2
  misc_feature 295..318: note = CDR3
  source 1..351: mol_type = other DNA
gaggtccagctgcaacaatctggacctgagctggtgaagcctggggcttcagtgaagatcgcctgtaaggcttctggatacacgttcacaggctactacatgaagtgggtgaagcagagccttggaaagagccttgagtggattggagatattaatcctaacaacggtggtactaactacaaccagcacttcaagggcaaggccacattgactgtagacgagtcctccagcacagcctacatggagctccgcagcctgacatctgaggactctgcagtctattactgtgcaagaggagtcgactggatttttgctttctggggccaagggactctggtcactgtctctcca

SEQ ID NO: 2 - protein, 117 aa, synthetic construct
  REGION 1..117: note = mAb heavy chain variable domain
  REGION 31..35: note = MISC_FEATURE - CDR1
  REGION 50..66: note = MISC_FEATURE - CDR2
  REGION 99..106: note = MISC_FEATURE - CDR3
EVQLQQSGPELVKPGASVKIACKASGYTFTGYYMKWVKQSLGKSLEWIGDINPNNGGTNYNQHFKGKATLTVDESSSTAYMELRSLTSEDSAVYYCARGVDWIFAFWGQGTLVTVSP

SEQ ID NO: 3 - protein, 5 aa, synthetic construct
  REGION 1..5: note = mAb43 heavy chain CDR1
GYYMK

SEQ ID NO: 4 - protein, 17 aa, synthetic construct
  REGION 1..17: note = mAb43 heavy chain CDR2
DINPNNGGTNYNQHFKG

SEQ ID NO: 5 - protein, 8 aa, synthetic construct
  REGION 1..8: note = mAb43 heavy chain CDR3
GVDWIFAF

SEQ ID NO: 6 - DNA, 321 bp, synthetic construct
  misc_feature 1..321: note = mAb43 light chain variable domain
  misc_feature 70..100: note = CDR1
  misc_feature 148..168: note = CDR2
  misc_feature 265..291: note = CDR3
  source 1..321: mol_type = other DNA
agtattgtgatgacccagactcccaaattcctgcctgtatcagcaggagacagggttaccatgacctgcaaggccagtcagagtgtgggtaataaagtagcctggtaccaacagaagccaggacagtctcctaaactgctgatatattatgcaaccaatcgctacactggagtccctgatcgcttcactggcagtggatctgggacagatttcactttcaccatcagcagtgtgcaggttgaagacctggcagtttatttctgtcagcagcattatagctctccgtggacgttcggtggagggaccaagctggaaatcaaa

SEQ ID NO: 7 - protein, 107 aa, synthetic construct
  REGION 1..107: note = mAb43 light chain variable domain
  REGION 24..34: note = MISC_FEATURE - CDR1
  REGION 50..56: note = MISC_FEATURE - CDR2
  REGION 89..97: note = MISC_FEATURE - CDR3
SIVMTQTPKFLPVSAGDRVTMTCKASQSVGNKVAWYQQKPGQSPKLLIYYATNRYTGVPDRFTGSGSGTDFTFTISSVQVEDLAVYFCQQHYSSPWTFGGGTKLEIK

SEQ ID NO: 8 - protein, 11 aa, synthetic construct
  REGION 1..11: note = mAb43 light chain CDR1
KASQSVGNKVA

SEQ ID NO: 9 - protein, 7 aa, synthetic construct
  REGION 1..7: note = mAb43 light chain CDR2
YATNRYT

SEQ ID NO: 10 - protein, 9 aa, synthetic construct
  REGION 1..9: note = mAb43 light chain CDR3
QQHYSSPWT

SEQ ID NO: 11 - protein, 468 aa, synthetic construct
  REGION 1..468: note = mAb20 heavy chain variable domain
MDMRVPAQLLGLLLLWLRGARCQIQLVQSGPELKKPGETVKISCKASGYIFTNYPMHWVKQAPGKGLKWMGWINTYSGVPTHADDFKGRFAFSLETSANSAFLQINNLKNEDTATYFCTRSRITPTGGYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 12 - protein, 7 aa, synthetic construct
  REGION 1..7: note = mAb20 heavy chain CDR1
GYIFTNY

SEQ ID NO: 13 - protein, 6 aa, synthetic construct
  REGION 1..6: note = mAb20 heavy chain CDR2
NTYSGV

SEQ ID NO: 14 - protein, 13 aa, synthetic construct
  REGION 1..13: note = mAb20 heavy chain CDR3
SRITPTGGYAMDY

SEQ ID NO: 15 - protein, 241 aa, synthetic construct
  REGION 1..241: note = mAb20 light chain variable domain
MDMRVPAQLLGLLLLWLRGARCDVVMTQTPLSLPVSLGDQASISCRSSQSLVHSNGNTYLHWYLQKPGQSPNLLISKVSNRFSGVPERFSGSGSGTDFTLKISRVEAEDLGVYFCSQNTHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 16 - protein, 16 aa, synthetic construct
  REGION 1..16: note = mAb20 light chain CDR1
RSSQSLVHSNGNTYLH

SEQ ID NO: 17 - protein, 7 aa, synthetic construct
  REGION 1..7: note = mAb20 light chain CDR2
KVSNRFS

SEQ ID NO: 18 - protein, 9 aa, synthetic construct
  REGION 1..9: note = mAb20 light chain CDR3
SQNTHVPWT

SEQ ID NO: 19 - protein, 117 aa, synthetic construct
  REGION 1..117: note = mAb43 heavy chain variable domain
  REGION 31..35: note = MISC_FEATURE - CDR1
  REGION 50..66: note = MISC_FEATURE - CDR2
  REGION 99..106: note = MISC_FEATURE - CDR3
QVQLQQSGPELVKPGASVKIACKASGYTFTGYYMKWVKQSLGKSLEWIGDINPNNGGTNYNQHFKGKATLTVDESSSTAYMELRSLTSEDSAVYYCARGVDWIFAFWGQGTLVTVSS

SEQ ID NO: 20 - protein, 233 aa, synthetic construct
  REGION 1..233: note = Light Chain: M43-hIgG1
  REGION 20..126: note = MISC_FEATURE - Variable Light (SEQ ID NO:7)
MLPSQLIGFLLLWVPASRGSIVMTQTPKFLPVSAGDRVTMTCKASQSVGNKVAWYQQKPGQSPKLLIYYATNRYTGVPDRFTGSGSGTDFTFTISSVQVEDLAVYFCQQHYSSPWTFGGGTKLEIKRTDAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 466 aa, synthetic construct
  REGION 1..466: note = Heavy Chain: M43-hIgG1
  REGION 20..136: note = MISC_FEATURE - Variable Heavy (SEQ ID NO:19)
MDWTWRILFLVAAATGAHSQVQLQQSGPELVKPGASVKIACKASGYTFTGYYMKWVKQSLGKSLEWIGDINPNNGGTNYNQHFKGKATLTVDESSSTAYMELRSLTSEDSAVYYCARGVDWIFAFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 462 aa, synthetic construct
  REGION 1..462: note = Heavy Chain: M43-hIgG2
  REGION 20..136: note = MISC_FEATURE - Variable Heavy (SEQ ID NO:19)
MDWTWRILFLVAAATGAHSQVQLQQSGPELVKPGASVKIACKASGYTFTGYYMKWVKQSLGKSLEWIGDINPNNGGTNYNQHFKGKATLTVDESSSTAYMELRSLTSEDSAVYYCARGVDWIFAFWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYNTTPPMLDSDGSFFLYSKLTVDKSRWQQGNVFSCSVMHEALHNHYTQKSLSLSPGK

SEQ ID NO: 23 - protein, 513 aa, synthetic construct
  REGION 1..513: note = Heavy Chain: M43-hIgG3
  REGION 20..136: note = MISC_FEATURE - Variable Heavy (SEQ ID NO:19)
MDWTWRILFLVAAATGAHSQVQLQQSGPELVKPGASVKIACKASGYTFTGYYMKWVKQSLGKSLEWIGDINPNNGGTNYNQHFKGKATLTVDESSSTAYMELRSLTSEDSAVYYCARGVDWIFAFWGQGTLVTVSSASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQFNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 24 - protein, 463 aa, synthetic construct
  REGION 1..463: note = Heavy Chain: M43-IgG4
  REGION 20..136: note = MISC_FEATURE - Variable Heavy (SEQ ID NO:19)
MDWTWRILFLVAAATGAHSQVQLQQSGPELVKPGASVKIACKASGYTFTGYYMKWVKQSLGKSLEWIGDINPNNGGTNYNQHFKGKATLTVDESSSTAYMELRSLTSEDSAVYYCARGVDWIFAFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 17 aa, synthetic construct
  REGION 1..17: note = GS+3xGGGGS linker
GSGGGGSGGGGSGGGGS

SEQ ID NO: 26 - protein, 15 aa, synthetic construct
  REGION 1..15: note = AVI tag sequence for site-directed biotinylation
GLNDIFEAQKIEWHE

SEQ ID NO: 27 - protein, 498 aa, synthetic construct
  REGION 1..498: note = Heavy Chain: hIgG1-GS-3XGGGGS-AVI
MDWTWRILFLVAAATGAHSQVQLQQSGPELVKPGASVKIACKASGYTFTGYYMKWVKQSLGKSLEWIGDINPNNGGTNYNQHFKGKATLTVDESSSTAYMELRSLTSEDSAVYYCARGVDWIFAFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGGGSGGGGSGGGGSGLNDIFEAQKIEWHE

SEQ ID NO: 28 - protein, 494 aa, synthetic construct
  REGION 1..494: note = Heavy Chain: M43-hIgG2-GS-3XGGGGS-AVI
MDWTWRILFLVAAATGAHSQVQLQQSGPELVKPGASVKIACKASGYTFTGYYMKWVKQSLGKSLEWIGDINPNNGGTNYNQHFKGKATLTVDESSSTAYMELRSLTSEDSAVYYCARGVDWIFAFWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYNTTPPMLDSDGSFFLYSKLTVDKSRWQQGNVFSCSVMHEALHNHYTQKSLSLSPGKGSGGGGSGGGGSGGGGSGLNDIFEAQKIEWHE

SEQ ID NO: 29 - protein, 545 aa, synthetic construct
  REGION 1..545: note = Heavy Chain: M43-hIgG3-GS-3XGGGGS-AVI
MDWTWRILFLVAAATGAHSQVQLQQSGPELVKPGASVKIACKASGYTFTGYYMKWVKQSLGKSLEWIGDINPNNGGTNYNQHFKGKATLTVDESSSTAYMELRSLTSEDSAVYYCARGVDWIFAFWGQGTLVTVSSASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQFNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGSGGGGSGGGGSGGGGSGLNDIFEAQKIEWHE

SEQ ID NO: 30 - protein, 495 aa, synthetic construct
  REGION 1..495: note = Heavy Chain: M43-hIgG4-GS-3XGGGGS-AVI
MDWTWRILFLVAAATGAHSQVQLQQSGPELVKPGASVKIACKASGYTFTGYYMKWVKQSLGKSLEWIGDINPNNGGTNYNQHFKGKATLTVDESSSTAYMELRSLTSEDSAVYYCARGVDWIFAFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSGGGGSGGGGSGGGGSGLNDIFEAQKIEWHE

SEQ ID NO: 31 - protein, 369 aa, Homo sapiens
  REGION 95..99: note = MISC_FEATURE - Extracellular Loop 1 (ECL1)
  REGION 169..175: note = MISC_FEATURE - Extracellular Loop 2 (ECL2)
  REGION 242..245: note = MISC_FEATURE - Extracellular Loop 3 (ECL3)
MEFLERTYLVNDKAAKMYAFTLESVELQQKPVNKDQCPRERPEELESGGMYHCHSGSKPTEKGANEYAYAKWKLCSASAICFIFMIAEVVGGHIAGSLAVVTDAAHLLIDLTSFLLSLFSLWLSSKPPSKRLTFGWHRAEILGALLSILCIWVVTGVLVYLACERLLYPDYQIQATVMIIVSSCAVAANIVLTVVLHQRCLGHNHKEVQANASVRAAFVHALGDLFQSISVLISALIIYFKPEYKIADPICTFIFSILVLASTITILKDFSILLMEGVPKSLNYSGVKELILAVDGVLSVHSLHIWSLTMNQVILSAHVATAASRDSQVVRREIAKALSKSFTMHSLTIQMESPVDQDPDCLFCEDPCD